(19) United States
(12) Patent Application Publication
(10) Pub. No.: US 2016/0145639 A1
(43) Pub. Date: May 26, 2016

(54) AGENT FOR IMPARTING RESISTANCE TO FEEDING DAMAGE BY PHYTOPHAGOUS ARTHROPOD (As Amended)

(71) Applicant: RIKEN, Saitama (JP)

(72) Inventors: Takeshi NAKANO, Saitama (JP); Tadao ASAMI, Saitama (JP); Ayumi YAMAGAMI, Saitama (JP)

(21) Appl. No.: 14/899,941

(22) PCT Filed: Jun. 19, 2014
(86) PCT No.: PCT/JP2014/066230

§ 371 Date: Dec. 18, 2015

(30) Foreign Application Priority Data
Jun. 21, 2013 (JP) 2013-130971

(51) Int. Cl. C12N 15/82; C07K 14/415

(52) U.S. Cl. CPC C12N 15/8286; C07K 14/415

(57) ABSTRACT

An object of the present invention is to develop and provide a method for enhancing the resistance of a plant itself to feeding damage by a phytophagous arthropod. A gene expression system containing, in an expressible state, a gene encoding a variant BIL1/BZR1 protein with a particular point mutation is transferred to a desired plant to thereby impart resistance to feeding damage by a phytophagous arthropod to the plant.

### TECHNICAL FIELD

The present invention relates to an agent for imparting resistance to feeding damage by a phytophagous arthropod, a method for imparting resistance to feeding damage by a phytophagous arthropod to a plant, and a plant resistant to feeding damage by a phytophagous arthropod.

### BACKGROUND ART

In the cultivation of crops, feeding damage by phytophagous arthropods, i.e., so-called agricultural pests, brings about serious problems, such as reduction in the yields of crops and the spread of plant diseases mediated by microbes or viruses, to the agricultural field. Hence, the control or extermination of agricultural pests is an agriculturally important challenge.

In the past, the application of chemical pesticides has mainly been practiced for the control or extermination of agricultural pests. The control using chemical pesticides, however, presents major problems such as the emergence of drug-resistant individuals, insecticidal action on useful insects, environmental pollutions, and residues on crops. Particularly, a shift to a sustained control technique in consonance with the environment has been demanded in response to increasing environmental consciousness in recent years. Thus, alternative control techniques for chemical pesticides have been received attention.

Typical examples thereof include biological control. The biological control is a method for controlling or exterminating agricultural pests, pathogenic microbes, or weed, etc., by use of natural enemies as biological pesticides (natural enemy preparations) based on a prey-predator relationship or a host-parasite relationship in the native ecosystem.

For example, Amblyseius swirskii and Orius strigicollis are used as natural enemies for biological control for thrips known as a difficult-to-control pest to cause serious damage to various crops. Amblyseius swirskii, however, is less active at low temperatures and preys thrips in reduced amounts, disadvantageously resulting in significant reduction in control effect. On the other hand, Orius strigicollis is effective in that it can be active at low temperatures and preys thrips in large amounts. However, its initial rate of colonization or rate of proliferation after pasturing is low (Non Patent Literature 1).

In order to solve this problem, a method exploiting insectary plants that promote the colonization or propagation of natural enemies for biological control has been developed (Non Patent Literature 2). The insectary plants, also called natural enemy-fostering plants, can enhance the rate of colonization or proliferation of natural enemies on crops by providing environmental conditions that facilitate the working of the natural enemies.

However, the introduction of insectary plants further increases cost for the biological control, which generally requires higher cost than that of the chemical control. In addition, the cultivation management of insectary plants themselves in addition to crops is necessary. Thus, there has arisen the new problem that a large amount of labor is required.

Hence, it has been desired to develop a novel technique that facilitates management at low cost and is capable of further effectively controlling or exterminating agricultural pests.

### CITATION LIST

### Non Patent Literature

Non Patent Literature 1: Kazuki Kakimoto et al., 2007, Japanese Journal of Applied Entomology and Zoology 51 (1): 29-37.
Non Patent Literature 2: Kazuya Nagai and Mitsuharu Hikawa, 2012, Japanese Journal of Applied Entomology and Zoology 56 (2): 57-64.

### SUMMARY OF INVENTION

### Technical Problem

An object of the present invention is to develop and provide a method for enhancing the resistance of a plant itself to feeding damage by a phytophagous arthropod without depending on chemical pesticides or biological pesticides.

### Solution to Problem

To attain the object, the present inventors have tackled the development of, not methods based on external factors such as chemical pesticides or biological pesticides, but a method for preventing feeding damage by agricultural pests by enhancing the properties of plants themselves, i.e., their pest-repellent effects. As a result, the present inventors have found that when a particular mutant gene of BIL1/BZR1 gene is expressed in plant cells, the plant acquires strong resistance to feeding damage by phytophagous arthropods such as thrips and whitefly known as a difficult-to-control pest. The BIL1/BZR1 protein is a protein that functions as a transcriptional factor downstream of the brassinosteroid signaling pathway (BR signaling pathway). Brassinosteroid is known as a plant hormone that is involved in plant growth regulation, photomorphogenesis, vascular bundle formation control, chloroplast functional regulation, etc. (Azpiroz R. et al., 1988, Plant Cell, 10: 219-230; Clouse S. & Sasse J., 1998, Annu. Rev. Plant Physiol. Plant Mol. Biol., 49: 427-45; Mandava N., 1988, Annu. Rev. Plant Physiol. Plant Mol. Biol., 39: 23-52; and Sakurai A. et al., 1999, Brassinosteroids, Steroidal Plant Hormones, Tokyo: Springer). Brassinosteroid has already been known to participate in the induction of disease resistance (brassinosteroid-mediated disease resistance; BDR) and impart plant disease resistance to plants by enhancing the intracellular signal transduction of brassinosteroid (Nakashita et al., 2003, Plant Journal, 33: 887-98). However, resistance to plant diseases caused by microbial infection and feeding damage control by repellent effects on phytophagous arthropods are considered to function under totally different mechanisms from the plant physiological standpoint. In fact, the BIL1/BZR1 protein is known to control development (Wang et al., 2002, Developmental Cell, 2: 505-513), but cannot induce plant disease resistance, unlike other brassinosteroid signaling factors (W02012/077786).

The present inventors have further found that when a bil1/bzr1 mutant gene of Arabidopsis thaliana of the family Brassicaceae is transferred to Lotus japonicus of the family Leguminosae or Solanum lycopersicum of the family Solanaceae, the resulting transformed plant can acquire resistance to feeding damage similar to that of Arabidopsis thaliana, and revealed that the bil1/bzr1 mutant gene can impart resistance to feeding damage by a phytophagous arthropod to a wide range of plants beyond species. The present invention is based on these new findings and provides the following:

(1) An agent for imparting resistance to feeding damage by a phytophagous arthropod, the agent comprising a polypeptide represented by any of the following amino acid sequences (a) to (c) or an active fragment thereof:

(a) a polypeptide in which proline (P) at position 234 in the amino acid sequence shown in SEQ ID NO: 1 is substituted by leucine (L),

(b) a polypeptide comprising an amino acid sequence derived from the polypeptide (a) by the deletion, substitution, or addition of one or several amino acids except for leucine at position 234, and

(c) a polypeptide having 60% or more amino acid identity to the polypeptide (a).

(2) An agent for imparting resistance to feeding damage by a phytophagous arthropod, the agent comprising a gene expression system containing, in an expressible state, a nucleic acid encoding a polypeptide represented by any of amino acid sequences (a) to (c) or an active fragment thereof according to (1).

(3) An agent for imparting resistance to feeding damage by a phytophagous arthropod, the agent comprising a gene expression system containing, in an expressible state, a nucleic acid represented by any of the following nucleotide sequences (d) to (g) or an active fragment thereof:

(d) a polynucleotide in which cytosine (C) at position 701 in the nucleotide sequence shown in SEQ ID NO: 2 is substituted by thymine (T),

(e) a polynucleotide comprising a nucleotide sequence derived from the polynucleotide (d) by the deletion, substitution, or addition of one or several bases except for thymine at position 701,

(f) a polynucleotide having 60% or more base identity to the polynucleotide (d), and

(g) a polynucleotide hybridizing under stringent conditions to a nucleotide sequence complementary to the polynucleotide (d).

(4) The agent for imparting resistance to feeding damage according to (2) or (3), wherein the gene expression system is overexpression type, constitutively active type, inducible expression type, or a combination thereof for the contained nucleic acid.

(5) The agent for imparting resistance to feeding damage according to any of (1) to (4), wherein the phytophagous arthropod is a phytophagous insect.

(6) The agent for imparting resistance to feeding damage according to (5), wherein the phytophagous insect is a species belonging to the order Thysanoptera or the family Aleyrodidae.

(7) The agent for imparting resistance to feeding damage according to any of (1) to (6), wherein the agent is intended for a dicotyledon.

(8) A method for imparting resistance to feeding damage by a phytophagous arthropod to a plant, the method comprising administering an agent for imparting resistance to feeding damage according to any of (1) to (7) to a desired plant.

(9) A plant resistant to feeding damage by a phytophagous arthropod and progeny thereof, the plant and the progeny each comprising a gene expression system containing, in an expressible state, a nucleic acid encoding a polypeptide represented by any of the following amino acid sequences (a) to (c) or an active fragment thereof:

(a) a polypeptide in which proline (P) at position 234 in the amino acid sequence shown in SEQ ID NO: 1 is substituted by leucine (L),

(b) a polypeptide comprising an amino acid sequence derived from the polypeptide (a) by the deletion, substitution, or addition of one or several amino acids except for leucine at position 234, and

(c) a polypeptide having 60% or more amino acid identity to the polypeptide (a).

(10) A plant resistant to feeding damage by a phytophagous arthropod and progeny thereof, the plant and the progeny each comprising a gene expression system containing, in an expressible state, a nucleic acid represented by any of the following nucleotide sequences (d) to (g) or an active fragment thereof:

(d) a polynucleotide in which cytosine (C) at position 701 in the nucleotide sequence shown in SEQ ID NO: 2 is substituted by thymine (T),

(e) a polynucleotide comprising a nucleotide sequence derived from the polynucleotide (d) by the deletion, substitution, or addition of one or several bases except for thymine at position 701,

(f) a polynucleotide having 60% or more base identity to the polynucleotide (d), and

(g) a polynucleotide hybridizing under stringent conditions to a nucleotide sequence complementary to the polynucleotide (d).

(11) The plant resistant to feeding damage and the progeny thereof according to (9) or (10), wherein the gene expression system is overexpression type, constitutively active type, inducible expression type, or a combination thereof for the contained nucleic acid.

(12) The plant resistant to feeding damage and the progeny thereof according to any of (9) to (11), wherein the phytophagous arthropod is a phytophagous insect.

(13) The plant resistant to feeding damage and the progeny thereof according to (12), wherein the phytophagous insect is a species belonging to the order Thysanoptera or the family Aleyrodidae.

(14) The plant resistant to feeding damage and the progeny thereof according to any of (9) to (13), wherein the desired plant is a dicotyledon.

The present specification encompasses the contents described in the specification and/or drawings of Japanese Patent Application No. 2013-130971, on which the priority of the present application is based.

### Advantageous Effects of Invention

The agent for imparting resistance to feeding damage according to the present invention can be administered to a desired plant to thereby impart resistance to feeding damage by a phytophagous arthropod to the plant.

The method for imparting resistance to feeding damage by a phytophagous arthropod to a plant according to the present invention is capable of conveniently imparting resistance to feeding damage by a phytophagous arthropod to a desired plant. As a result, a plant resistant to feeding damage by a phytophagous arthropod can be easily produced.

The plant resistant to feeding damage and the progeny thereof according to the present invention can provide a plant that has acquired sustained resistance to feeding damage by a phytophagous arthropod, without the need of special management or cost.

### BRIEF DESCRIPTION OF DRAWINGS

FIG. 1 is a diagram showing the alignment of the amino acid sequences of Arabidopsis thaliana BIL1/BZR1 protein (AtBIL1/BZR1) and an AtBIL1/BZR1 protein ortholog of each plant species in the particular mutation of the present invention and its neighboring regions. In the AtBIL1/BZR1 protein ortholog, portions having amino acids identical to those of the AtBIL1/BZR1 protein after the alignment are indicated by asterisks. The hyphens represent gaps. The numeric value on the left side of each amino acid sequence represents the position of the N-terminal amino acid from initiating methionine defined as position 1 in each full-length protein represented by SEQ ID NO on the right side of each amino acid sequence. A P234L-AtBIL1/BZR1 protein in which the boxed proline (P) corresponding to position 234 in the AtBIL1/BZR1 protein was point-mutated to leucine (L), and an ortholog mutated therefrom are variant BIL1/BZR1 proteins which are subject to the present invention.

FIG. 2 is a diagram showing that an Arabidopsis thaliana bil1-1D strain with a P234L-Atbil1 gene, which corresponds to the plant resistant to feeding damage of the present invention, has resistance to feeding damage by Thrips tabaci. After an experiment, the number of leaves found to have feeding signs per strain was counted. The test strains were classified according to the number of leaves that suffered feeding damage, and evaluated for their feeding damage statuses on the basis of the ratio of each group to the total number of test strains.

FIG. 3 is a diagram showing that a gene expression system containing a P234L-Atbil1 gene, which is the agent for imparting resistance to feeding damage according to the present invention, was administered to a wild strain of Lotus japonicus different from Arabidopsis thaliana that was a species of the origin of the P234L-Atbil1 gene, and the obtained transgenic Lotus japonicus Lj-Atbil1-OX strain acquired resistance to feeding damage by Thrips tabaci.

FIG. 4 is a diagram showing that an Arabidopsis thaliana bil1-1D strain with a P234L-Atbil1 gene, which corresponds to the plant resistant to feeding damage of the present invention, has resistance to feeding damage by Frankliniella occidentalis. In order to show the feeding damage status, a wild strain (for a control) and the bil1-1D strain were photographed from above after an experiment.

FIG. 5 is a diagram showing that a gene expression system containing a P234L-Atbil1 gene was administered to a wild strain of Solanum lycopersicum, and the obtained transgenic Solanum lycopersicum Sl-Atbil1-OX strain acquired resistance to feeding damage by Bemisia tabaci.

### DESCRIPTION OF EMBODIMENTS

1. Agent for Imparting Resistance to Feeding Damage

1-1. Summary

The first aspect of the present invention is an agent for imparting resistance to feeding damage. The agent for imparting resistance to feeding damage according to this aspect is a preparation that can be administered to a desired plant to thereby impart resistance to feeding damage by a phytophagous arthropod to the plant.

1-2. Definition

In the present specification, the “resistance to feeding damage” refers to the property of being insusceptible or less susceptible to feeding damage by a phytophagous arthropod through a repellent effect on the phytophagous arthropod.

In the present specification, the “phytophagous arthropod” refers to an arthropod that ingests a living plant body. In the present specification, the phytophagous arthropod particularly corresponds to an agricultural pest that causes damage to various crops including vegetables, fruit trees, flowers, and tea. Specific examples of the phytophagous arthropod include phytophagous insects and species belonging to the family Tetranychidae. The phytophagous insects which are subject to the present invention include, for example, species belonging to the order Thysanoptera, species belonging to the suborder Heteroptera, species belonging to the superfamily Aphidoidea, species belonging to the family Aleyrodidae, species belonging to the superfamily Coccoidea, species belonging to the suborder Symphyta, and species belonging to the order Lepidoptera. Species belonging to the order Thysanoptera and species belonging to the family Aleyrodidae known as difficult-to-control pests are preferred.

In the present specification, the species belonging to the order Thysanoptera include, for example, Thrips tabaci, Frankliniella occidentalis, Frankliniella intonsa, Henothrips haemorrhoidalis, Thrips palmi, Thrips nigropilosus, and Scirtothrips dorsalis.

In the present specification, the species belonging to the suborder Heteroptera include Megacopta punctatissima, Nezara antennata, Halyomorpha halys, Eurydema rugosa, Glaucias subpunctatus, Stephanitis pyrioides, Stephanitis nashi, Stephanitis typica, and Galeatus spinifrons.

In the present specification, the species belonging to the superfamily Aphidoidea include, for example, Aphis gossypii, Aphis glycines, Aphis craccivora, Acyrthosiphon pisum, Aphis forbesi, Aphis spiraecola, Myzus persicae, Rhodobium porosum, Rhopalosiphum rufiabdominalis, Brevicoryne brassicae, Lipaphis erysimi ,Neotoxoptera formosana, Uroleucon formosanum, Chaetosiphon fragaefolii, Macrosiphum euphorbiae, Rhopalosiphum maidis, Rhopalosiphum padi, Sitobion akebiae, Aulacorthum solani, Toxoptera citricida, Ovatus malisuctus, and Hyalopterus pruni.

In the present specification, the species belonging to the family Aleyrodidae include, for example, Bemisia tabaci, Bemisia argentifolii, Trialeurodes vaporariorum, and Aleurocanthus spiniferus .

In the present specification, the species belonging to the superfamily Coccoidea include, for example, Icerya purchasi Maskell, Ceroplastes rubens, and Eulecanium kunoense.

In the present specification, the species belonging to the family Tetranychidae include, for example, Tetranychus urticae, Tetranychus kanzawai, Amphitetranychus viennensis, Panonychus citri, Panonychus ulmi, and Bryobia praetiosa.

The site to be ingested by the phytophagous arthropod in the plant body is not limited. The site to be ingested may be, for example, any of leaves, flowers, stems, roots, shoots, fruits, and seeds. The form of ingestion by the phytophagous arthropod is not limited. The form of ingestion may be the direct ingestion of the plant body or may be the sucking of a plant body fluid through the insertion of snout or the like into the plant body.

In the present specification, the “administration to a desired plant” refers to the contact or introduction of the agent for imparting resistance to feeding damage with or to the desired plant to which resistance to feeding damage is to be imparted, when the agent for imparting resistance to feeding damage comprises a particular variant BIL1/BZR1 protein or an active fragment thereof. Specific examples of the contact or the introduction include nebulization, spraying, coating, and dipping. Alternatively, the “administration to a desired plant” refers to the introduction of a gene expression system into the desired plant cells, when the agent for imparting resistance to feeding damage comprises the gene expression system. The desired plant will be described in detail in the second aspect, so that the description thereof is omitted here.

1-3. Configuration

The agent for imparting resistance to feeding damage according to this aspect comprises a particular variant BIL1/BZR1 protein or an active fragment thereof, or a gene expression system containing, in an expressible state, a nucleic acid encoding the protein or the active fragment.

(1) Particular Variant BIL1/BZR1 Protein or Active Fragment Thereof

The “BIL1/BZR1 protein” is a factor that functions as a downstream transcriptional factor in the intracellular brassinosteroid signaling pathway. BZR1 is a synonym for BIL1. In the present specification, only BIL1 shall therefore be used below.

The “particular variant BIL1 protein” is a variant BIL1 protein having a point mutation by which proline (P) at position 234 with initiating methionine defined as position 1 is substituted by leucine (L), for example, in the case of an amino acid sequence constituting the Arabidopsis thaliana wild-type BIL1 protein (hereinafter, also referred to as an “AtBIL1 protein”) shown in SEQ ID NO: 1. In the present specification, this point mutation is referred to as “P234L”, and a variant AtBIL1 protein having P234L is indicated by a “P234L-AtBIL1 protein”. This P234L point mutation in the AtBIL1 protein is a gain-of-function mutation that stabilizes the AtBIL1 protein and brings about the high accumulation thereof in cells. Although the specific mechanism is unknown, Examples mentioned later have demonstrated that a plant expressing the P234L-AtBIL1 protein can acquire resistance to feeding damage by a phytophagous arthropod.

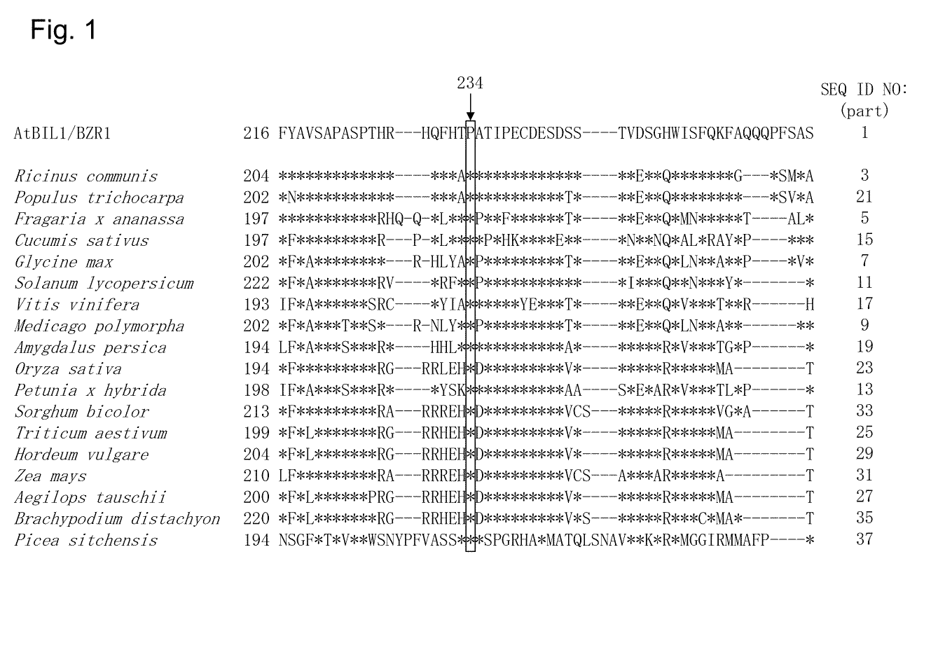

The “particular variant BIL1 protein” may be not only the P234L-AtBIL1 protein but a variant BIL1 protein having a mutation equivalent to P234L in any of AtBIL1 protein orthologs of other plant species. The “AtBIL1 protein orthologs” refer to a group of proteins having the same functions as those of the AtBIL1 protein in other plant species. The “mutation equivalent to P234L” refers to a point mutation by which P that corresponds to position 234 of the AtBIL1 protein when the amino acid sequences of each AtBIL1 protein ortholog and the AtBIL1 protein are aligned, and is highly conserved among species (FIG. 1) is substituted by L. Specific examples thereof include a polypeptide in which P at position 221 in Ricinus communis BIL1protein (RcBIL1 protein) shown in SEQ ID NO: 3 is substituted by L (P221L-RcBIL1 protein), a polypeptide in which P at position 216 in Fragaria×ananassa BIL1 protein (FaBIL1 protein) shown in SEQ ID NO: 5 is substituted by L (P216L-FaBIL1 protein), a polypeptide in which P at position 219 in Glycine max BIL1 protein (GmBIL1 protein) shown in SEQ ID NO: 7 is substituted by L (P219L-GmBIL1 protein), a polypeptide in which P at position 219 in Medicago polymorpha BIL1 protein (MpBIL1 protein) shown in SEQ ID NO: 9 is substituted by L (P219L-MpBIL1 protein), a polypeptide in which P at position 239 in Solanum lycopersicum BIL1 protein (SlBIL1 protein) shown in SEQ ID NO: 11 is substituted by L (P239L-S1BIL1 protein), a polypeptide in which P at position 215 in Petunia×hybrida BIL1 protein (PhBIL1 protein) shown in SEQ ID NO: 13 is substituted by L (P215L-PhBIL1 protein), a polypeptide in which P at position 214 in Cucumis sativus BIL1 protein (CsBIL1 protein) shown in SEQ ID NO: 15 is substituted by L (P214L-CsBIL1 protein), a polypeptide in which P at position 210 in Vitis vinifera BIL1 protein (VvBIL1 protein) shown in SEQ ID NO: 17 is substituted by L (P210L-VvBIL1 protein), a polypeptide in which P at position 211 in Amygdalus persica BIL1 protein (ApBIL1 protein) shown in SEQ ID NO: 19 is substituted by L (P211L-ApBIL1 protein), a polypeptide in which P at position 219 in Populus trichocarpa BIL1 protein (PtBIL1 protein) shown in SEQ ID NO: 21 is substituted by L (P219L-PtBIL1 protein), a polypeptide in which P at position 212 in Oryza sativa BIL1 protein (OsBIL1 protein) shown in SEQ ID NO: 23 is substituted by L (P212L-OsBIL1 protein), a polypeptide in which P at position 217 in Triticum aestivum BIL1 protein (TaBIL1 protein) shown in SEQ ID NO: 25 is substituted by L (P217L-TaBIL1 protein), a polypeptide in which P at position 218 in Aegilops tauschii BIL1 protein (AetBIL1 protein) shown in SEQ ID NO: 27 is substituted by L (P218L-AetBIL1 protein), a polypeptide in which P at position 222 in Hordeum vulgare BIL1 protein (HvBIL1 protein) shown in SEQ ID NO: 29 is substituted by L (P222L-HvBIL1 protein), a polypeptide in which P at position 228 in Zea mays BIL1 protein (ZmBIL1 protein) shown in SEQ ID NO: 31 is substituted by L (P228L-ZmBIL1 protein), a polypeptide in which P at position 231 in Sorghum bicolor BIL1 protein (SbBIL1 protein) shown in SEQ ID NO: 33 is substituted by L (P231L-SbBIL1 protein), a polypeptide in which P at position 238 in Brachypodium distachyon BIL1 protein (BdBIL1 protein) shown in SEQ ID NO: 35 is substituted by L (P238L-BdBIL1 protein), and a polypeptide in which P at position 215 in Picea sitchensis BIL1 protein (PsBIL1 protein) shown in SEQ ID NO: 38 is substituted by L (P215L-PsBIL1 protein). In the present specification, these variant BIL1 proteins are referred to as “P234L-AtBIL1 protein orthologs”.

The “particular variant BIL1 protein” may have the deletion, substitution, or addition of one or more or several amino acids within a range that maintains the activity of resistance to feeding damage by a phytophagous arthropod, in addition to P234L in the AtBIL1 protein or a point mutation of a P234L counterpart in the P234L-AtBIL1 protein ortholog. In the present specification, the term “several” refers to 2 to 10, 2 to 7, 2 to 5, or 2 to 4. Furthermore, the particular variant BIL1 protein may be a polypeptide that retains P234L of the AtBIL1 protein or a point mutation of a P234L counterpart of the P234L-AtBIL 1 protein ortholog and has 60% or more, 70% or more, 80% or more, 85% or more, 90% or more, 92% or more, 95% or more, 98% or more, or 99% or more amino acid identity to the BIL1 protein. In this context, the “amino acid identity” refers to the ratio (%) of the number of identical amino acid residues in the amino acid sequence of a subject polypeptide to the total number of amino acid residues in the amino acid sequence of the BIL1 protein when these two amino acid sequences are arranged (aligned) with the highest amino acid identity therebetween in the presence or absence of introduced gap(s).

The “active fragment thereof” refers to a polypeptide fragment of the P234L-AtBIL1 protein or the P234L-AtBIL1 protein ortholog mentioned above and is a polypeptide fragment that retains P234L of the AtBIL1 protein or a point mutation of a P234L counterpart of the P234L-AtBIL1 protein ortholog and maintains the activity of resistance to feeding damage by a phytophagous arthropod.

(2) Particular bil1 Mutant Gene

The “nucleic acid encoding the particular variant BIL1 protein or the active fragment thereof” refers to a bil1 mutant gene encoding the particular variant BIL1 protein mentioned above, or a gene fragment encoding the active fragment of the variant BIL1 protein. In this context, examples of the particular bil1 mutant gene include a gene encoding the P234L-AtBIL1 protein. Specifically, the particular bil1 mutant gene is a bil1 mutant gene having at least a point mutation by which cytosine (c) at position 701 with a start codon adenine (a) defined as position 1 in the nucleotide sequence of the Arabidopsis thaliana wild-type BIL1 gene (AtBIL1 gene) shown in SEQ ID NO: 2 is substituted by thymine (t) (this point mutation is referred to as “c701t”). This mutant gene encodes the P234L-AtBIL1 protein. In the present specification, the mutant gene is therefore referred to as a “P234L-Atbil1 gene”.

The “particular bil1 mutant gene” may be not only the P234L-Atbil1 gene but a bil1 mutant gene encoding the P234L-AtBIL1 protein ortholog. Specific examples thereof include a bil1 mutant gene in which c at position 662 in Ricinus communis wild-type BIL1 gene (RcBIL1 gene) shown in SEQ ID NO: 4 is substituted by t (P221L-Rcbil1 gene), a bil1 mutant gene in which c at position 647 in Fragaria×ananassa BIL1 gene (FaBIL1 gene) shown in SEQ ID NO: 6 is substituted by t (P216L-Fabil1 gene), a bil1 mutant gene in which c at position 656 in Glycine max BIL1 gene (GmBIL1 gene) shown in SEQ ID NO: 8 is substituted by t (P219L-Gmbil1 gene), a bil1 mutant gene in which c at position 656 in Medicago polymorpha BIL1 gene (MpBIL1 gene) shown in SEQ ID NO: 10 is substituted by t (P219L-Mpbil1 gene), a bil1 mutant gene in which c at position 716 in Solanum lycopersicum BIL1 gene (SlBIL1 gene) shown in SEQ ID NO: 12 is substituted by t (P239L-Slbil1 gene), a bil1 mutant gene in which c at position 644 in Petunia×hybrida BIL1 gene (PhBIL1 gene) shown in SEQ ID NO: 14 is substituted by t (P215L-Phbil1 gene), a bil1 mutant gene in which c at position 641 in Cucumis sativus BIL1 gene (CsBIL1 gene) shown in SEQ ID NO: 16 is substituted by t (P214L-Csbil1 gene), a bil1 mutant gene in which c at position 629 in Vitis vinifera BIL1 gene (VvBIL1 gene) shown in SEQ ID NO: 18 is substituted by t (P210L-Vvbil1 gene), a bil1 mutant gene in which c at position 632 in Amygdalus persica BIL1 gene (ApBIL1 gene) shown in SEQ ID NO: 20 is substituted by t (P211L-Apbil1 gene), a bil1 mutant gene in which c at position 656 in Populus trichocarpa BIL1 gene (PtBIL1 gene) shown in SEQ ID NO: 22 is substituted by t (P219L-Ptbil1 gene), a bil1 mutant gene in which c at position 635 in Oryza sativa BIL1 gene (OsBIL1 gene) shown in SEQ ID NO: 24 is substituted by t (P212L-Osbil1 gene), a bil1 mutant gene in which c at position 650 in Triticum aestivum BIL1 gene (TaBIL1 gene) shown in SEQ ID NO: 26 is substituted by t (P217L-Tabil1 gene), a bil1 mutant gene in which c at position 653 in Aegilops tauschii BIL1 gene (AetBIL1 gene) shown in SEQ ID NO: 28 is substituted by t (P218L-Aetbil1 gene), a bil1 mutant gene in which c at position 665 in Hordeum vulgare BIL1 gene (HvBIL1 gene) shown in SEQ ID NO: 30 is substituted by t (P222L-Hvbil1 gene), a bil1 mutant gene in which c at position 683 in Zea mays BIL1 gene (ZmBIL1 gene) shown in SEQ ID NO: 32 is substituted by t (P228L-Zmbil1 gene), a bil1 mutant gene in which c at position 692 in Sorghum bicolor BIL1 gene (SbBIL1 gene) shown in SEQ ID NO: 34 is substituted by t (P231L-Sbbil1 gene), a bil1 mutant gene in which c at position 713 in Brachypodium distachyon BIL1 gene (BdBIL1 gene) shown in SEQ ID NO: 36 is substituted by t (P238L-Bdbil1 gene), and a bil1 mutant gene in which c at position 644 in Picea sitchensis BIL1 gene (PsBIL1 gene) shown in SEQ ID NO: 38 is substituted by t (P215L-Psbil1 gene). In the present specification, these bil1 mutant genes are referred to as “P234L-Atbil1 gene orthologs”.

The “particular bil1 mutant gene” may have the deletion, substitution, or addition of one or more or several bases within a range in which the variant BIL1 protein encoded thereby maintains the activity of resistance to feeding damage by a phytophagous arthropod, in addition to c701t in the P234L-Atbil1 gene or a point mutation of a c701t counterpart in the P234L-Atbil1 gene ortholog. Alternatively, the particular bil1 mutant gene may be a polynucleotide that retains c701t of the P234L-Atbil1 gene or a point mutation of a c701t counterpart of the P234L-Atbil1 gene ortholog and has 60% or more, 70% or more, 80% or more, 85% or more, 90% or more, 92% or more, 95% or more, 98% or more, or 99% or more base identity to the bil1 gene. In this context, the “base identity” refers to the ratio (%) of the number of identical bases in the nucleotide sequence of a subject polynucleotide to the total number of bases in the nucleotide sequence of the bil1 gene when these two nucleotide sequences arraigned with the highest base identity therebetween in the presence or absence of introduced gap(s). Furthermore, the particular bil1 mutant gene includes a nucleic acid that hybridizes under stringent conditions to a nucleic acid fragment comprising a nucleotide sequence complementary to a partial nucleotide sequence of the wild-type BIL1 gene, retains the c701t point mutation of the P234L-Atbil1 gene or a point mutation of a c701t counterpart of the P234L-Atbil1 gene ortholog, and encodes a polypeptide that maintains the activity of resistance to feeding damage by a phytophagous arthropod. The “stringent conditions” mean conditions under which no nonspecific hybrid is formed. The stringent conditions are conditions involving hybridization followed by washing, for example, with 0.1×SSC and 0.1% SDS at 65° C.

In the present specification, the “nucleic acid” mainly refers to a natural nucleic acid such as DNA and/or RNA and can also include an artificially chemically modified or constructed nucleic acid or nucleic acid analog. The nucleic acid may be labeled, if necessary, at its phosphate group, sugar, and/or base with a labeling material for nucleic acids.

(3) Gene Expression System

The “gene expression system” refers to one expression system unit that allows a gene or a fragment thereof incorporated therein to be expressed. The gene expression system constituting the agent for imparting resistance to feeding damage according to this aspect contains, in an expressible state, the nucleic acid encoding the particular variant BIL 1 protein or the active fragment thereof mentioned above.

In the present specification, the “expressible state” means that the contained nucleic acid, i.e., the nucleic acid encoding the particular variant BIL 1 protein or the active fragment thereof, is inserted in the gene expression system such that the nucleic acid can be expressed. Specifically, the expressible state means that the nucleic acid is placed under the control of a promoter and a terminator in the gene expression system. Thus, the gene expression system has at least a promoter and a terminator in addition to the particular bil1 mutant gene or the fragment thereof.

The promoter contained in the gene expression system is not particularly limited by its type as long as the promoter has transcriptional control functions in plant cells. A promoter known in the art can be used. Examples thereof include cauliflower mosaic virus (CaMV)-derived 35S promoter, Ti plasmid-derived nopaline synthase gene promoter Pnos, Zea mays-derived ubiquitin promoter, Oryza sativa-derived actin promoter, Nicotiana tabacum-derived PR protein promoter, ribulose bisphosphate carboxylase small subunit (Rubisco ssu) promoter, and histone promoter. All of these promoters are suitable as a promoter for a gene expression system having the properties of overexpression type and inducible expression type in combination mentioned later.

The terminator contained in the gene expression system is not particularly limited by its type as long as the terminator has transcriptional termination functions in plant cells. Examples thereof include nopaline synthase (NOS) gene terminator, octopine synthase (OCS) gene terminator, CaMV 35S terminator, E. coli lipopolyprotein lpp 3′ terminator, trp operon terminator, amyB terminator, and ADH1 gene terminator.

The gene expression system constituting the agent for imparting resistance to feeding damage according to this aspect can selectively contain other gene expression regulation regions, in addition to the promoter or the terminator. Other gene expression regulation regions correspond to, for example, an enhancer, a poly-A addition signal, a 5′-UTR (untranslated region) sequence, a tag or selective marker gene, a multicloning site, and a replication origin.

Examples of the enhancer contained in the gene expression system include an enhancer region containing an upstream sequence in CaMV 35S promoter. Examples of the tag or selective marker gene include drug resistance genes (e.g., tetracycline resistance gene, ampicillin resistance gene, kanamycin resistance gene, hygromycin resistance gene, spectinomycin resistance gene, chloramphenicol resistance gene, and neomycin resistance gene), fluorescent or luminescent reporter genes (e.g., luciferase, β-galactosidase, β-glucuronidase (GUS), and green fluorescent protein (GFP) genes), and enzyme genes such as neomycin phosphotransferase II (NPT II) gene, dihydrofolate reductase gene, and blasticidin S resistance gene. These gene expression regulation regions are not particularly limited by their types as long as the regions can exert specific functions in plant cells. Those known in the art can be appropriately selected according to the plant to be transfected.

The gene expression system constituting the agent for imparting resistance to feeding damage according to this aspect can control the expression of the contained nucleic acid as overexpression type, constitutively active type, inducible expression type, multicopy type, or combined type thereof

The “overexpression-type gene expression system” is a gene expression system that allows the contained nucleic acid, i.e., the particular bil1 mutant gene or the fragment thereof, to be overexpressed. This gene expression system achieves the expression of the contained particular bil1 mutant gene or fragment thereof at 2 times or more, preferably 5 times or more, more preferably 10 times or more or 20 times or more the ordinary expression level of the wild-type BIL1 gene per cell of each plant species.

The “constitutively active type gene expression system” is a gene expression system that allows the contained nucleic acid, i.e., the particular bil1 mutant gene or the fragment thereof, to be constitutively expressed. This gene expression system achieves the constant and continuous expression of the particular bil1 mutant gene or the fragment thereof, regardless of the time of expression or expression sites.

The “inducible expression-type gene expression system” is a gene expression system that can induce the expression of the contained nucleic acid, i.e., the particular bil1 mutant gene or the fragment thereof This gene expression system achieves the time-specific or site-specific expression of the contained particular bil1 mutant gene or fragment thereof.

The “multicopy-type gene expression system” is a gene expression system that can produce a plurality of copies through its own high ability to replicate after being transferred to plant cells so that the number of the gene expression system is increased per plant cell. This gene expression system is advantageous in that the gene expression system can increase the number of the gene expression system itself and thereby increase an expression level per cell as a whole, even if the expression level of the particular bil1 mutant gene or the fragment thereof from each individual gene expression system is low.

The “combined-type gene expression system” is a gene expression system having the combined properties of these gene expression systems. Examples thereof include gene expression systems having the combined properties of the overexpression type and the constitutively active type, the overexpression type and the inducible expression type, or the overexpression type, the constitutively active type, and the multicopy type. For example, the combined-type gene expression system of the overexpression type and the constitutively active type can contain the 35S promoter mentioned above and thereby allows the contained particular bil1 mutant gene or fragment thereof to be constitutively overexpressed.

Specific examples of the gene expression system mentioned above include expression vectors. For example, a plasmid expression vector based on a plasmid or a viral expression vector based on a virus is suitable as the gene expression system.

When the gene expression system constituting the agent for imparting resistance to feeding damage according to this aspect is a plasmid expression vector, for example, a binary vector of pPZP series, pSMA series, pUC series, pBR series, pBluescript series (Stratagene Corp.), pTriEXTM series (Takara Bio Inc.), pBI series, pRI series, or pGW series can be used as a core moiety serving as a backbone.

When the gene expression system constituting the agent for imparting resistance to feeding damage according to this aspect is a viral expression vector, for example, cauliflower mosaic virus (CaMV), bean golden mosaic virus (BGMV), or tobacco mosaic virus (TMV) can be used as a virus moiety.

2. Method for Imparting Resistance to Feeding Damage

2-1. Summary

The second aspect of the present invention is a method for imparting resistance to feeding damage by a phytophagous arthropod to a desired plant. In the method of this aspect, the agent for imparting resistance to a plant according to the first aspect can be administered to a desired plant to thereby easily impart resistance to feeding damage by a phytophagous arthropod to the plant.

2-2. Method

The method for imparting resistance to feeding damage according to this aspect comprises an administration step as an essential step and also comprises a regeneration step as an optional step. Hereinafter, each step will be described.

(Administration Step)

In the present specification, the “administration step” refers to administering the agent for imparting resistance to feeding damage according to the first aspect to a desired plant. The “desired plant” is a plant of interest to which resistance to feeding damage by a phytophagous arthropod is to be imparted, and is a recipient plant of the agent for imparting resistance to feeding damage. The recipient plant according to this aspect is not particularly limited and may be any of angiosperms and gymnosperms. The angiosperms encompass all of dicotyledons and monocotyledons. Typical examples thereof include agriculturally important plants, for example, crop plants such as cereals, flowers, vegetables, and fruits. Specifically, the dicotyledons correspond to species belonging to the family Brassicaceae (e.g., Brassica oleracea var. capitata, Raphanus sativus var. longipinnatus, Brassica rapa var. pekinensis, and Brassica napus), species belonging to the family Leguminosae (e.g., Glycine max, Arachis hypogaea, Pisum sativum L., Phaseolus vulgaris L., Vigna angularis, Vicia faba, and Lathyrus odoratus), species belonging to the family Solanaceae (e.g., Solanum lycopersicum, Solanum melongena, Solanum tuberosum L., Nicotiana tabacum, Capsicum annuum L. Grossum, Capsicum annuum, and Petunia×hybrida), species belonging to the family Rosaceae (e.g., Fragaria×ananassa, Malus pumila, Pyrus pyrifolia, Amygdalus persica, Eriobotrya japonica, Prunus dulcis, Prunus salicina, Rosa spp., Prunus mume, and Cerasus spp.), species belonging to the family Cucurbitaceae (e.g., Cucumis sativus, Cucurbitaceae Juss., Cucurbita spp., Cucumis melo L., Citrullus lanatus, and Luffa cylindrica), species belonging to the family Liliaceae (e.g., Allium fistulosum, Allium cepa, and Lilium L.), the family Rutaceae (e.g., Citrus unshiu, Citrus sinensis, Citrus×paradisi, Citrus Limon, and Citrus junos), species belonging to the family Vitaceae (e.g., Vitis vinifera), species belonging to the family Theaceae (Camellia sinensis), and the family Compositae (e.g., Lactuca sativa). The monocotyledons correspond to species belonging to the family Poaceae (e.g., Oryza sativa, Triticum aestivum, Hordeum vulgare, Zea mays, Saccharum officinarum, Sorghum bicolor, and Sorghum bicolor).

The agent for imparting resistance to feeding damage according to the first aspect comprises the gene expression system. The agent for imparting resistance to feeding damage, used in this aspect, is the gene expression system for a plant as a host. Thus, the administration method in this step can be carried out using a method known in the art to be able to transfer the gene expression system into plant cells and transform the recipient plant.

When the agent for imparting resistance to feeding damage is constituted by a plasmid expression vector, for example, a protoplast method, a particle gun method, or an Agrobacterium method can be used as a suitable transformation method.

The “protoplast method” is a method for using plant cells (protoplasts) whose cell walls were removed by enzymatic treatment with cellulase or the like to transfer the agent for imparting resistance to feeding damage into the plant cells. This method can be further classified according to a gene transfer method into an electroporation method, a microinjection method, and a polyethylene glycol method, etc. The electroporation method is a method for applying electric pulse to a mixed solution of protoplasts and the agent for imparting resistance to feeding damage to transfer the gene into the protoplasts. The microinjection method is a method for directly transferring the agent for imparting resistance to feeding damage into protoplasts under a microscope using a microneedle. The polyethylene glycol method is a method for transferring the agent for imparting resistance to feeding damage into protoplasts by the action of polyethylene glycol.

The “particle gun method” is a method for attaching the agent for imparting resistance to feeding damage to fine particles of gold, tungsten, or the like and injecting the resulting particles into plant tissue cells with high-pressure gas to transfer the agent for imparting resistance to feeding damage into the cells. Transformed cells harboring the gene of interest in the genomic DNA of the host plant cells can be obtained. The transformed cells are usually selected according to a product of the marker gene in the agent for imparting resistance to feeding damage.

The “Agrobacterium method” is a method for transforming plant cells using a bacterium of the genus Agrobacterium (e.g., A. tumefaciens and A. rhizogenes) and a Ti plasmid derived therefrom as a transforming factor. This method can transfer the agent for imparting resistance to feeding damage into the genomic DNA of the host plant cells.

All of these methods are methods known in the art. For the details thereof, see appropriate protocols described in, for example, Handbook of Plant Metabolic Engineering (2002, NTS Inc.) or Shinban Modem Syokubutsu No Jikken Protokoru (New Edition Experimental Protocols for Model Plants in English): from genetic approaches to genomic analysis (2001, Gakken Medical Shujunsha Co., Ltd.).

When the agent for imparting resistance to feeding damage is a viral expression vector (e.g., cauliflower mosaic virus (CaMV), bean golden mosaic virus (BGMV), or tobacco mosaic virus (TMV)), the plant cells of interest can be infected with the agent for imparting resistance to feeding damage to obtain transformed cells. For the details of such a gene transfer method using the viral vector, see, for example, the method of Hohn et al. (Molecular Biology of Plant Tumors (Academic Press, New York) 1982, pp. 549) or the specification of U.S. Pat. No. 4,407,956.

In this step, the plant species of the origin of the particular bil1 mutant gene contained in the agent for imparting resistance to feeding damage does not have to be the same as the recipient plant species of the agent for imparting resistance to feeding damage. For example, the agent for imparting resistance to feeding damage, containing a P234L-Atbil1 mutant gene derived from Arabidopsis thaliana, may be administered to a plant of the family Leguminosae. This is because, as shown in Example 2 mentioned later, the agent for imparting resistance to feeding damage according to the first aspect can impart resistance to feeding damage to a recipient plant beyond plant species.

The state of the plant when the agent for imparting resistance to feeding damage is transferred thereto differs depending on the transformation method used. For example, the protoplast method is applied to the single cell state of a plant, whereas the particle gun method, the Agrobacterium method, or the method using a viral expression vector can be applied to any of single cell, tissue, callus, and plant individual states of a plant. A method for directly transferring the gene expression system to the cells of a plant individual is also called in planta method, which is excellent in that the method can produce transgenic plants without the need of the regeneration step mentioned later. For the details of the in planta method, see Murakami et al., 2013, Plant Cell Physiol, 54: 518-527.

(Regeneration Step)

In the present specification, the “regeneration step” refers to tissue-culturing the plant cell given the agent for imparting resistance to feeding damage in the administration step to regenerate a plant individual. This step is an optional step that is carried out when the state of the plant given the agent for imparting resistance to feeding damage is other than a plant individual (i.e., single cell, tissue, and callus). This step may comprise dedifferentiating the plant cell according to the need, culturing the undifferentiated cell to form callus, and regenerating a transgenic plant from the callus.

Examples of the specific regeneration method in the regeneration step include an in vitro regeneration method which involves dedifferentiating the protoplast given the agent for imparting resistance to feeding damage by the protoplast method, culturing the undifferentiated cell, and regenerating a plant body through callus. This method is known in the art. See, for example, the aforementioned Handbook of Plant Metabolic Engineering (2002, NTS Inc.) or Shinban Moderu Syokubutsu No Jikken Protokoru (New Edition Experimental Protocols for Model Plants in English): from genetic approaches to genomic analysis (2001, Gakken Medical Shujunsha Co., Ltd.). A plant hormone such as auxin, gibberellin, and/or cytokinin may be used for promoting the proliferation and/or division of transformed cells.

3. Plant Resistant to Feeding Damage and Progeny Thereof

3-1. Summary

The third aspect of the present invention is a plant resistant to feeding damage and progeny thereof. The plant resistant to feeding damage and the progeny thereof according to this aspect achieve the sustained and effective control of feeding damage by a phytophagous arthropod at low cost without the need of labors other than usual plant cultivation management.

3-2. Configuration

The “plant resistant to feeding damage” of this aspect has substantially the same configuration as that of the transgenic plant obtained by the method for imparting resistance to feeding damage according to the second aspect using the agent for imparting resistance to feeding damage according to the first aspect. Specifically, a first-generation transgenic plant obtained by the method of the second aspect is the plant resistant to feeding damage. In the present specification, the “first-generation transgenic plant” encompasses clones having the same genetic information thereas. The first-generation transgenic plant corresponds to, for example, a cutting, a grafting, or a layering of a portion of a plant body collected from the first-generation transgenic plant, a plant body regenerated via callus formation after cell culture, or a new trophozoite resulting from a vegetative reproductive organ (e.g., rhizomes, tuberous roots, corms, and runners) obtained by asexual reproduction from the first-generation transgenic plant.

The plant resistant to feeding damage of this aspect is a plant containing at least one gene expression system derived from the agent for imparting resistance to feeding damage according to the first aspect.

The configuration of the gene expression system carried by the plant resistant to feeding damage and the progeny thereof according to this aspect is as described in the first aspect, so that the specific description thereof is omitted here.

The “progeny thereof” is progeny of the plant resistant to feeding damage. Specifically, the progeny thereof refers to offspring obtained via the sexual reproduction of the transgenic plant (first generation) obtained by the method for imparting resistance to feeding damage according to the second aspect, and is an individual that retains the gene expression system derived from the agent for imparting resistance to feeding damage according to the first aspect. The progeny thereof corresponds to, for example, a seedling of the first-generation transgenic plant.

### EXAMPLE

### Example 1

### Verification of Resistance to Feeding Damage by Phytophagous Arthropod in Plant Resistant to Feeding Damage (1)

(Purpose)

The plant resistant to feeding damage of the present invention was examined for its resistance to feeding damage by a phytophagous arthropod.

(Material)

Arabidopsis thaliana was used as a recipient plant. A bil1-1D strain was used as the plant resistant to feeding damage of the present invention, and ecotype Columbia (Col-0) was used as a control wild strain (WT). The bil1-1D strain is a gain-of-function mutant that has the P234L-Atbil1 gene and stabilizes the BIL1 protein resulting in the high accumulation thereof.

Adults of Thrips tabaci were used as the phytophagous arthropod.

(Method)

(1) Cultivation of Arabidopsis thaliana

Each Arabidopsis thaliana was inoculated onto a medium containing ½ Murashige & Skoog (MS) medium (Duchefa Biochemie BV.), 0.8% phyto agar (Duchefa Biochemie B.V.), and 1.5% sucrose, and cultured on the medium for 10 days after germination. Then, each seedling was transferred to culture soil in a pot and cultivated under long-day conditions of 16 hours under a white light at 22° C. and 8 hours in the dark.

(2) Feeding Damage Assay of Thrips tabaci

The adults of Thrips tabaci were pastured for 2 months in a mesh cage (50 cm×50 cm×50 cm) at 25° C. The pots of a plurality of the wild strains and a plurality of the bil1-1D strains of Arabidopsis thaliana cultured for 3 weeks in culture soil were alternately placed in the cage. This alternate placement is intended to reduce a positional error in which Thrips tabaci is biased towards the wild strains or the bil1 strains. The pots in the cage were cultivated for 2 weeks. Then, the number of leaves found to have feeding signs per strain was counted to evaluate the feeding damage status.

(Results)

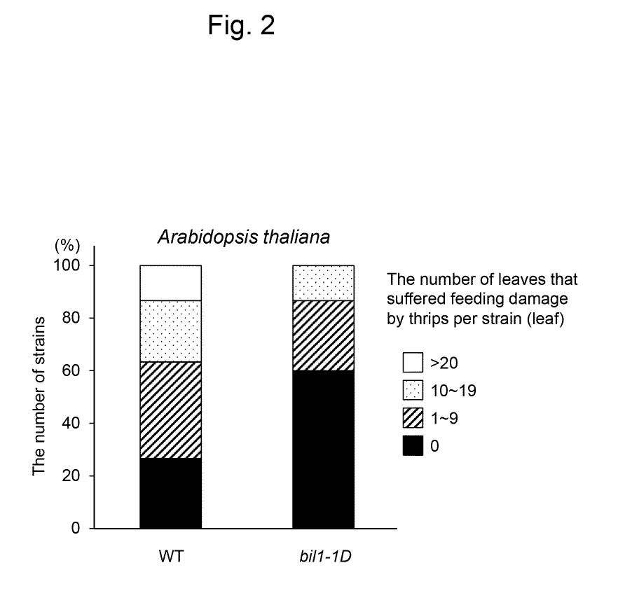

The results are shown in FIG. 2. A strain group having no leaf that suffered feeding damage (which corresponds to “0” in the graph) was less than 30% of all of the wild strains (WT), but occupied 60% or more of the bil1-1D strains, demonstrating difference by more than double therebetween. These results indicate that the plant resistant to feeding damage of the present invention has resistance to feeding damage by a phytophagous arthropod.

### Example 2

### Verification of Effect of Imparting Resistance to Feeding Damage by Agent for Imparting Resistance to Feeding Damage—(1)

(Purpose)

The administration of the agent for imparting resistance to feeding damage according to the present invention to a desired plant was confirmed to allow the plant to acquire resistance to feeding damage by a phytophagous arthropod.

(Material)

A Lotus japonicus ecotype Gifu strain was used as a wild strain serving as a recipient plant.

As in Example 1, imagos of Thrips tabaci were used as the phytophagous arthropod.

(Method)

(1) Preparation of Agent for Imparting Resistance to Feeding Damage According to Present Invention

A gene expression vector containing the P234L-Atbil1 gene, constituting the agent for imparting resistance to feeding damage according to the present invention, was constructed. P234L-Atbil1 cDNA was prepared by PCR from the cDNA of the Arabidopsis thaliana bil1-1D strain having the P234L point mutation. The primers used were P234L-Atbil1 -Forward (5′-CACCATGACTTCGGATGGAGCTAC-3′: SEQ ID NO: 39) and P234L-Atbil1-Reverse 5′-TCAACCACGAGCCTTCCCAT-3′: SEQ ID NO: 40). The obtained amplification product was inserted to pENTR/D-TOPO (Life Technologies, Inc.), cloned, and then integrated to a binary vector pGWB2 (Nakagawa et al., 2007, J Biosci Bioeng, 104: 34-41) containing CaMV 35S promoter by the Gateway method to obtain a gene expression vector p35-P234L-Atbil1 constituting the agent for imparting resistance to feeding damage according to the present invention.

(2) Preparation of Transgenic Lotus japonicus Lj-Atbil1-OX Strain

The p35-P234L-Atbil1 constituting the agent for imparting resistance to feeding damage according to the present invention was administered to wild strains of Lotus japonicus by the method for imparting resistance to feeding damage. Specifically, p35-P234L-Atbil1 was first transferred to Agrobacterium tumefaciens C58 strains. The Agrobacterium transformants were introduced to the hypocotyls of Lotus japonicus ecotype Gifu strains by the in planta method according to a somewhat modification of the method of Murakami et al. (Murakami et al., 2013, Plant Cell Physiol, 54: 518-527). The transgenic strains were identified by PCR assay. The primers used for the PCR assay were a forward primer (5′-CGACACACTTGTCTACTCCA-3′: SEQ ID NO: 41) and a reverse primer (5′-CCCAACCAGCTTCAACACAA-3′: SEQ ID NO: 42) for P234L-Atbil1 detection. The reaction was carried out by a routine method. Strains confirmed to have the amplification product were used as transgenic Lotus japonicus Lj-Atbil1-OX strains.

(3) Cultivation of Lotus japonicus

The sterilized seeds of each ecotype Gifu strain were inoculated onto a medium containing ½ Murashige & Skoog (MS) medium (Duchefa Biochemie B.V.), 0.8% phyto agar (Duchefa Biochemie B.V.), and 1.5% sucrose and cultured on the medium for 1 week after germination. Then, each seedling was transferred to culture soil in a pot and cultivated under long-day conditions of 16 hours under a white light at 25° C. and 8 hours in the dark.

(4) Feeding Damage Assay of Thrips tabaci

The basic operation followed Example 1 provided that, in this Example, a plurality of the wild strains of Lotus japonicus cultured for 40 days in culture soil and a plurality of the transgenic Lotus japonicus Lj-Atbil1-OX strains prepared above were alternately placed in the cage.

(Results)

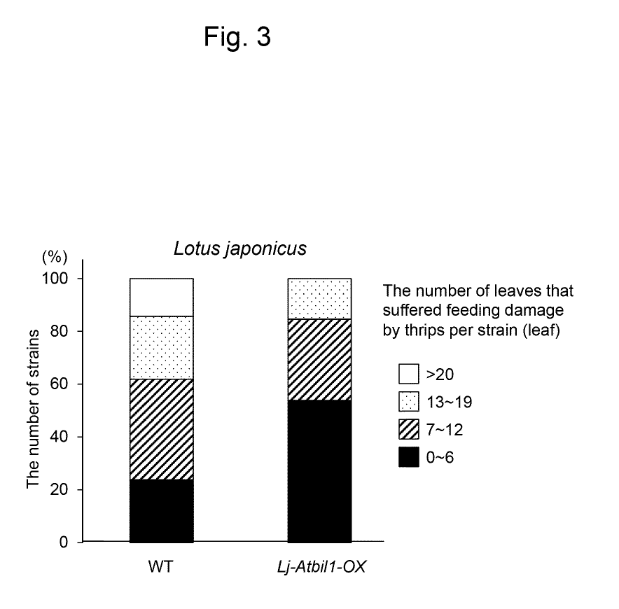

The results are shown in FIG. 3. A strain group having 0 to 6 leaves that suffered feeding damage (which corresponds to “0-6” in the graph) was less than 30% of all of the wild strains (WT), but occupied 50% or more of the transgenic Lotus japonicus Lj-Atbil1-OX strains harboring the gene expression system containing the P234L-Atbil1 gene, demonstrating difference as in Example 1.

The P234L-Atbil1 gene contained in the gene expression system used here was derived from Arabidopsis thaliana, which is a plant of the family Brassicaceae, whereas the plant to which this gene expression system was transferred was Lotus japonicus, which is a plant of the family Leguminosae. Thus, the results of this Example demonstrated that the agent for imparting resistance to feeding damage according to the present invention can impart resistance to feeding damage by a phytophagous arthropod to a recipient plant beyond plant species.

### Example 3

### Verification of Resistance to Feeding Damage by Phytophagous Arthropod in Plant Resistant to Feeding Damage—(2)

(Purpose)

The plant resistant to feeding damage of the present invention was examined for its resistance to feeding damage by a phytophagous arthropod different from that in Examples 1 and 2.

(Material and Method)

The basic materials and method followed Example 1, so that only points different from the description of Example 1 will be described here. In this Example, adults of Frankliniella occidentalis were used instead of Thrips tabaci used as the phytophagous arthropod in Examples 1 and 2. The feeding damage by Frankliniella occidentalis was evaluated by adjacently placing 6 pots each of the wild strains and the bil1-1D strains of Arabidopsis thaliana cultured for 3 weeks, in a cage of pastured Frankliniella occidentalis, and cultivating the plants for 2 weeks, followed by the visual observation of the feeding damage status.

(Results)

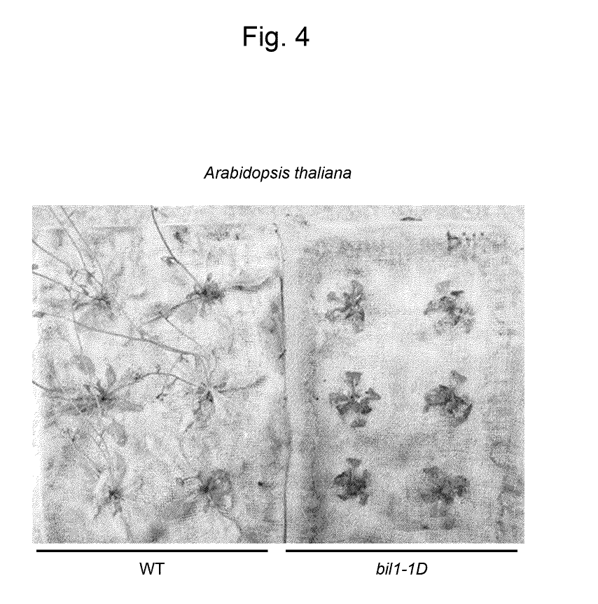

The results are shown in FIG. 4. In general, leaves that have suffered feeding damage by occidentalis have feeding signs of white small spots. The further progression of the feeding damage results in the whitening of whole leaves. As shown in FIG. 4, the resistance to feeding damage by Frankliniella occidentalis was also found to differ significantly between the wild strains and the bil1-1D strains. Specifically, all leaves of the 6 wild strains (WT) for a control were almost whitened and had faint color due to severe feeding damage by Frankliniella occidentalis, whereas almost all leaves of the 6 bil1-1D strains still had dark color without feeding signs. These results indicate that the plant resistant to feeding damage of the present invention has resistance to feeding damage by not only the particular phytophagous arthropod but various types of phytophagous arthropods.

### Example 4

### Verification of Effect of Imparting Resistance to Feeding Damage by Agent for Imparting Resistance to Feeding Damage—(2)

(Purpose)

The agent for imparting resistance to feeding damage according to the present invention was examined for its versatility for recipient plants and phytophagous arthropods to be controlled.

(Material)

Solanum lycopersicum belonging to the family Solanaceae was selected as a recipient plant. In this Example, a dwarf cultivar Micro-Tom for research was used as a wild strain.

Adults of Bemisia tabaci belonging to the family Aleyrodidae of the order Hemiptera were used as the phytophagous arthropod.

(Method)

(1) Preparation of Agent for Imparting Resistance to Feeding Damage of Present Invention

p35-P234L-Atbil1 constructed in Example 2 was used as a gene expression vector containing the P234L-Atbil1 gene, constituting the agent for imparting resistance to feeding damage according to the present invention.

(2) Preparation and Cultivation of Transgenic Solanum lycopersicum Sl-Atbil1-OX Strain

p35-P234L-Atbil1 was administered to wild strains of Solanum lycopersicum by the method for imparting resistance to feeding damage. The administration method, the confirmation of transgenic strains, and the cultivation of the transgenic strains were carried out according to the methods described in Example 2.

(4) Feeding Damage Assay of Bemisia tabaci

The wild strains of Solanum lycopersicum and three different lines of the Sl-Atbil1-OX strains (11-6-3 strain, 15-7-1 strain, and 25-1-2 strain) were each inoculated to culture soil and then cultured for 8 weeks. The resulting pots were placed in a mesh cage (50 cm×50 cm×50 cm) at 25° C., and approximately 100 adults of Bemisia tabaci were pastured for 1 month in the cage. Then, the number of Bemisia tabaci individuals observed in the back of the leaf per leaf in each strain was counted to evaluate the resistance to feeding damage.

(Results)

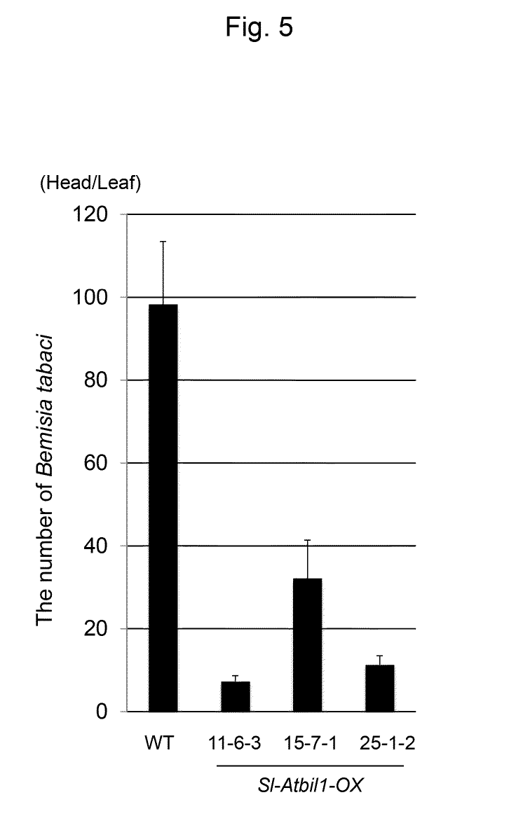

The results are shown in FIG. 5. As compared with the wild strains, all of the lines of the transgenic Solanum lycopersicum Sl-Atbil1-OX strains given the agent for imparting resistance to feeding damage according to the present invention were confirmed to have remarkable resistance to feeding damage by Bemisia tabaci.

The recipient plant used in this Example was Solanum lycopersicum, which is a plant of the family Solanaceae, not a plant of the family Brassicaceae or a plant of the family Leguminosae. Thus, the results of this Example were able to demonstrate again that the agent for imparting resistance to feeding damage according to the present invention can impart resistance to feeding damage by a phytophagous arthropod to a recipient plant beyond plant species, as verified in Example 2.

Moreover, the phytophagous arthropod used in this Example was Bemisia tabaci, which is different from the insects of the order Thysanoptera used in Examples 1 to 3. Whitefly is an insect belonging to the family Aleyrodidae of the order Hemiptera, which differs taxonomically in order from thrips. These results suggest that a plant transformed with the agent for imparting resistance to feeding damage according to the present invention can acquire resistance to feeding damage by various phytophagous arthropods.

All publications, patents, and patent applications cited herein are incorporated herein by reference in their entirety.

1. An agent for imparting resistance to feeding damage by a phytophagous arthropod, comprising a polypeptide represented by any of the following amino acid sequences (a) to (c) or an active fragment thereof:
(a) a polypeptide in which proline (P) at position 234 in the amino acid sequence shown in SEQ ID NO: 1 is substituted by leucine (L),
(b) a polypeptide comprising an amino acid sequence derived from the polypeptide (a) by the deletion, substitution, or addition of one or several amino acids except for leucine at position 234, and
(c) a polypeptide having 60% or more amino acid identity to the polypeptide (a).

2. An agent for imparting resistance to feeding damage by a phytophagous arthropod, comprising a gene expression system containing, in an expressible state, a nucleic acid encoding a polypeptide represented by any of amino acid sequences (a) to (c) or an active fragment thereof according to claim 1.

3. An agent for imparting resistance to feeding damage by a phytophagous arthropod, comprising a gene expression system containing, in an expressible state, a nucleic acid represented by any of the following nucleotide sequences (d) to (g) or an active fragment thereof:
(d) a polynucleotide in which cytosine (C) at position 701 in the nucleotide sequence shown in SEQ ID NO: 2 is substituted by thymine (T),
(e) a polynucleotide comprising a nucleotide sequence derived from the polynucleotide (d) by the deletion, substitution, or addition of one or several bases except for thymine at position 701,
(f) a polynucleotide having 60% or more base identity to the polynucleotide (d), and
(g) a polynucleotide hybridizing under stringent conditions to a nucleotide sequence complementary to the polynucleotide (d).

4. The agent for imparting resistance to feeding damage according to claim 2, wherein the gene expression system is overexpression type, constitutively active type, inducible expression type, or a combination thereof for the contained nucleic acid.

5. The agent for imparting resistance to feeding damage according to claim 1, wherein the phytophagous arthropod is a phytophagous insect.

6. The agent for imparting resistance to feeding damage according to claim 5, wherein the phytophagous insect is a species belonging to the order Thysanoptera or the family Aleyrodidae.

7. The agent for imparting resistance to feeding damage according to claim 1, wherein the agent is intended for a dicotyledon.

8. A method for imparting resistance to feeding damage by a phytophagous arthropod to a plant, comprising administering an agent for imparting resistance to feeding damage according to claim 1 to a desired plant.

9. A plant resistant to feeding damage by a phytophagous arthropod and progeny thereof, comprising a gene expression system containing, in an expressible state, a nucleic acid encoding a polypeptide represented by any of the following amino acid sequences (a) to (c) or an active fragment thereof:
(a) a polypeptide in which proline (P) at position 234 in the amino acid sequence shown in SEQ ID NO: 1 is substituted by leucine (L),
(b) a polypeptide comprising an amino acid sequence derived from the polypeptide (a) by the deletion, substitution, or addition of one or several amino acids except for leucine at position 234, and
(c) a polypeptide having 60% or more amino acid identity to the polypeptide (a).

10. A plant resistant to feeding damage by a phytophagous arthropod and progeny thereof, comprising a gene expression system containing, in an expressible state, a nucleic acid represented by any of the following nucleotide sequences (d) to (g) or an active fragment thereof:
(d) a polynucleotide in which cytosine (C) at position 701 in the nucleotide sequence shown in SEQ ID NO: 2 is substituted by thymine (T),
(e) a polynucleotide comprising a nucleotide sequence derived from the polynucleotide (d) by the deletion, substitution, or addition of one or several bases except for thymine at position 701,
(f) a polynucleotide having 60% or more base identity to the polynucleotide (d), and
(g) a polynucleotide hybridizing under stringent conditions to a nucleotide sequence complementary to the polynucleotide (d).

11. The plant resistant to feeding damage and the progeny thereof according to claim 9, wherein the gene expression system is overexpression type, constitutively active type, inducible expression type, or a combination thereof for the contained nucleic acid.

12. The plant resistant to feeding damage and the progeny thereof according to claim 9, wherein the phytophagous arthropod is a phytophagous insect.

13. The plant resistant to feeding damage and the progeny thereof according to claim 12, wherein the phytophagous insect is a species belonging to the order Thysanoptera or the family Aleyrodidae.

14. The plant resistant to feeding damage and the progeny thereof according to any claim 9, wherein the desired plant is a dicotyledon.


SEQUENCE LISTING (42 sequences)

SEQ ID NO: 1 - protein, 336 aa, Arabidopsis thaliana
MTSDGATSTSAAAAAAAAAAARRKPSWRERENNRRRERRRRAVAAKIYTGLRAQGDYNLPKHCDNNEVLKALCVEAGWVVEEDGTTYRKGCKPLPGEIAGTSSRVTPYSSQNQSPLSSAFQSPIPSYQVSPSSSSFPSPSRGEPNNNMSSTFFPFLRNGGIPSSLPSLRISNSCPVTPPVSSPTSKNPKPLPNWESIAKQSMAIAKQSMASFNYPFYAVSAPASPTHRHQFHTPATIPECDESDSSTVDSGHWISFQKFAQQQPFSASMVPTSPTFNLVKPAPQQMSPNTAAFQEIGQSSEFKFENSQVKPWEGERIHDVGMEDLELTLGNGKARG

SEQ ID NO: 2 - DNA, 1011 bp, Arabidopsis thaliana
atgacttcggatggagctacgtcgacatcagcagctgcagctgcggcggcggcagcagcggcgaggaggaagccgtcgtggagagaaagggagaataatcggaggagagaaagacggagaagagctgtagctgcgaagatatacactgggcttagagctcaaggtgattataatttgcctaaacattgtgataataatgaagtccttaaagctctttgtgttgaagctggttgggttgttgaagaagatggtactacttatcgcaagggatgcaagcctttacctggtgagatagctgggacttcatctcgagtaactccatattcatcacagaaccagagccctctttcatcagcctttcaaagtcccatcccatcttaccaagttagcccgtcttcttcatcattcccgagtccttctcgcggtgaaccaaataacaacatgtcctctacattcttccctttcctcagaaatggtggcattccttcttctcttccttccctcagaatctcaaacagttgtccagttaccccaccggtctcatcgccgacttctaagaacccgaaaccgttgcctaactgggaatctatcgctaagcaatccatggccattgctaaacaatcaatggcgtcttttaattatcctttctatgcggtttctgcacctgctagtccgacacatcgccaccagtttcataccccggctactatacctgaatgtgatgagtctgactcttccactgttgattctggtcattggataagctttcagaagtttgcacaacaacagccattctctgcctctatggtgccaacctctcctaccttcaatcttgtgaaacctgcgcctcagcagatgtctccaaatactgctgccttccaagagattggtcaaagctctgagtttaaatttgagaatagccaagttaaaccctgggaaggagagaggatacatgatgtgggtatggaggatcttgagcttacacttggaaatgggaaggctcgtggttga

SEQ ID NO: 3 - protein, 321 aa, Ricinus communis
MTSDGATSTSAAAAAARRKPSWRERENNRRRERRRRAIAAKIFSGLRAQGNYNLPKHCDNNEVLKALCSEAGWVVEDDGTTYRKGCRPPPIDIVGTSARITPYSSQNPSPLSSAFPSPIPSYQVSPSSSSFPSPTRGDNNHNNAASSILPFLQNAIPASLPPLRISNSAPVTPPLSSPTSRNPKPIPNWEFIAKQSMASFNYPFYAVSAPASPTHRQFHAPATIPECDESDSSTVESGQWISFQKFGPSMAAAMPTSPTYNLMKPVAEQILSSNVIKENGRSMEFEFGNGNGQVKPWEGERIHEVGLDDLELTLGNGKARS

SEQ ID NO: 4 - DNA, 966 bp, Ricinus communis
atgacgtcggacggagctacatcgacgtcagctgcagctgcagcagccaggaggaagccgtcctggagagaaagagagaataataggagaagagagagaaggagaagagcaattgctgctaaaatattttcaggattaagagctcaaggtaattataatttgcctaaacactgtgataataatgaggttttgaaagctctctgttctgaagctggttgggttgttgaagatgatggtactacttatcgcaagggatgcaggccacccccaattgatattgtaggcacttctgcaagaattactccatactcttcacaaaatccaagtccattatcttcagcatttcctagtccaattccttcttatcaagtcagtccctcttcctcttcattccctagccccactcgaggtgataataatcataataatgctgcatccagtatccttcctttcctccaaaatgccattcctgcatctctcccacctctccgaatttccaacagtgcacctgtgactccacctctttcatccccaacctcaagaaatcctaagccaattcccaattgggagttcattgcgaaacaatctatggcctcatttaattaccctttttatgctgtatctgccccagctagcccaactcaccgtcagtttcatgctcctgctactatacctgaatgtgatgagtctgattcatccactgtcgagtccggtcaatggataagctttcaaaaatttgggccttctatggctgctgcaatgccaacttctcctacctataatctcatgaaacctgtggctgagcaaattttgtccagtaatgtgattaaagagaatgggaggagtatggagtttgagtttggaaatgggaatggacaagtgaagccctgggaaggtgagaggattcatgaggtaggattggatgatctagagctcacacttggaaatgggaaggctcgaagttag

SEQ ID NO: 5 - protein, 314 aa, Fragaria x ananassa
MTSDGATSASTVRRKPSWRERENNRRRERRRRAIAAKIFAGLRAQGSFNLPKHCDNNEVLKALCAEAGWTVEEDGTTYRKGFRPPPVDTPGTSTKITPYSSLNQSPLSSSFPSPIPSYQASPSSSSFPSPSRCFDANNSSNLFPHLRSAIPSLPPLRISNSAPVTPPLSSPNSRKSQPIPNWDSIAKQSMASFNYPFYAVSAPASPTRHQQQLHTPPTIFECDESDTSTVESGQWMNFQKFATALSARPTSPTFNLVKPATAQQNLPANGMVEMGRGPEFEFLGGQVKPWVGERIHEVGLDDLELTLGNGKPRS

SEQ ID NO: 6 - DNA, 945 bp, Fragaria x ananassa
atgacgtcagacggagccacctcggcgtcgacggtgaggagaaagccgtcgtggagagaaagagagaacaacaggaggagagagaggagaaggagggcgattgcggccaagatattcgccgggcttcgagctcaggggagcttcaacttgcccaagcactgcgataacaacgaggtcttgaaggctctctgcgccgaggctggttggactgtcgaagaagacggcaccacttaccgaaagggattcaggccacccccagttgatacaccaggcacttcaaccaaaattacaccctattcttctctaaatcagagtcccttgtcttcttccttcccaagcccaattccttcctaccaagccagtccctcatcctcgtcttttccaagcccttctcgttgttttgatgccaacaattcatcaaatctctttccccatctgcgatctgctataccatctcttcctcctctccggatttctaacagtgctcctgtgacccctccactctcatctcccaactcaagaaagagccaaccaattcccaattgggactccattgccaaacaatccatggcctctttcaattaccctttttacgccgtgtctgctccagctagtccaactcgtcaccaacagcaactccatactcctcccaccatttttgaatgtgatgagtctgatacctcgactgttgaatctggccaatggatgaacttccagaaatttgcaaccgccttgtctgcaaggccaacctcccctaccttcaatcttgtgaaacctgccactgctcagcaaaacttgccagccaatggaatggtggagatggggagaggaccagagtttgagtttctcgggggacaagtgaagccgtgggtcggggagaggattcatgaagtgggattggatgatttagagctcacacttggcaatggtaagcctcgaagttaa

SEQ ID NO: 7 - protein, 320 aa, Glycine max
MTSDGATSAATHRRKPSWRERENNRRRERRRRAIAAKIYSGLRAQGNYNLPKHCDNNEVLKALCAEAGWTVEEDGTTYRKGCRAPLPGDGVGTSTRNTPFSSQNPSPLSSSFPSPIPSYQVSPSSSSFPSPSRLDANNPSNLIPYIRHAFPSSLPPLRISNSAPVTPPLSSPTSRNPKPIPTWDSIAKASMASSFNHSHHPFFAASAPASPTHRHLYAPPTIPECDESDTSTVESGQWLNFQAFAPSVSAVPISPTMNFIKPVVSQQHKHNLNLSGNGIQEMRISEPEFAMQVKPWVGERIHEVGLDDLELTLGSGKTPA

SEQ ID NO: 8 - DNA, 963 bp, Glycine max
atgacttcggacggagcaacgtcggcggcgacgcatcggaggaaaccttcgtggagggagagagagaacaaccggaggagagagaggagaaggagagccattgcagccaagatatactccggccttagggcgcaagggaactacaatttgcccaaacactgcgacaacaatgaggtcttgaaggctctctgcgccgaagctggttggaccgtcgaagaagacggaaccacctatcgcaagggatgcagggcaccacttcctggtgatggtgtaggcacctccaccagaaacactcctttctcatcgcaaaatccgagtcctctttcttcgtcatttccaagtccaattccttcatatcaagtgagcccttcctcctcgtctttcccgagcccgtctcgtttggatgcaaacaacccttcaaacctgattccttacattcggcatgcgtttccctcgtctctccctcctttgaggatatcaaacagcgcccctgttaccccgcctctgtcctcgccaacttcgcgaaaccccaagccaattcccacatgggattccattgccaaagcctctatggcatcatccttcaaccacagccaccaccctttcttcgcggcttcagcaccggctagccccacacaccgccacctttatgccccacccactatcccggagtgtgatgagtctgatacctccactgttgagtctggtcagtggctgaacttccaagcatttgcaccttctgtttctgctgtgcctatctctcccaccatgaactttatcaaaccagttgtgagtcagcagcacaagcacaacctcaatctgtctggtaacggaatccaagagatgagaatttcggagcccgaatttgcgatgcaggtgaagccctgggttggggagaggattcatgaagtggggttggatgatttggaactcacacttggaagtgggaagacgcctgcttag

SEQ ID NO: 9 - protein, 315 aa, Medicago polymorpha
MASDGATSAANSSRRKPSWRERENNRRRERRRRAIAAKIYAGLRSQGNYNLPKHCDNNEVLKALCAEAGWTVEEDGTTYRRGSRAETPGDGAGNFNRNNPFSSQNLSPLSSSFPSPIPSYQVSPSSSSFPSPSRMDANNNASNYIPYARTMFPNMSLPPLRISNSAPVTPPVSSPTSRNPKPMIPTWESIAKASGTSFNHPFFAASAPTSPSHRNLYTPPTIPECDESDTSTVESGQWLNFQAFAASAKSVSPTLNFMKPVINEQHNMLPHNRMQEMRISEPEFGVQVKPWVGERIHEVGLDDLELTLGNGKAPS

SEQ ID NO: 10 - DNA, 948 bp, Medicago polymorpha
atggcttctgacggagcaacttcggcggcgaattcaagtcgtcggaagccgtcgtggagggagagagagaacaacaggaggagagagagacggaggagagctatagcggcaaagatttacgcgggattaaggtctcaggggaattataatttaccaaaacactgtgataacaatgaggtcttgaaagctctttgtgctgaagctggttggactgttgaagaagatggcaccacttatcgcaggggatcaagggcagaaacaccaggcgatggtgcaggaaatttcaacagaaacaacccattttcatctcaaaatctaagtcctctttcatcatcatttccaagtccaatcccttcctaccaagttagcccctcttcctcttcattcccgagcccgtctcgtatggatgcaaacaacaatgcatcaaattacattccatatgctcgcaccatgttccccaacatgtctctcccacctttgagaatatcaaacagcgcgcccgtgactccacctgtctcatcaccaacctcaagaaatcctaaacccatgattccaacatgggaatcaattgccaaagcatcagggacatccttcaatcaccctttctttgcagcttctgcgcctactagtccatcccaccggaacctttatactccacctactattcctgaatgtgatgagtctgatacttctactgttgagtctggacagtggttgaatttccaagcctttgcagcttctgctaaatcagtctctcctacattgaactttatgaaacctgtaattaatgagcagcataacatgctgcctcataacagaatgcaggagatgaggatttcagagccagagtttggggtgcaggtcaagccttgggttggggaaaggattcatgaagtgggcttggatgatttggagcttactcttggaaatggaaaggcacctagttaa

SEQ ID NO: 11 - protein, 333 aa, Solanum lycopersicum
MMWEAGESPASSSAGAGAGGSGGAGVGLPESGGGGGGGRRKPSWRERENNRRRERRRRAVAAKIYTGLRAQGNYNLPKHCDNNEVLKALCTEAGWIVEPDGTTYRKGCKPTPMEIGGTSTNITPSSSRHPSPPSSYFASPIPSYQPSPTSSSFPSPSRADANMLSHPYSFLQNVVPSSLPPLRISNSAPVTPPLSSPTRHPKQTFNLETLAKESMFALNIPFFAASAPASPTRVQRFTPPTIPECDESDSSTIDSGQWINFQKYASNVPPSPTFNLVKPVPQPLRPNDMITDKGKSIDFDFENVSVKAWEGERIHDVGFDDLELTLGSGNARI

SEQ ID NO: 12 - DNA, 1002 bp, Solanum lycopersicum
atgatgtgggaagctggagaatcaccagcatcttcttcggccggtgccggagctggtggaagtggaggtgccggagttggtttaccggaaagtggtggtggtggtggtggtgggagaaggaaaccatcatggagagaaagagagaataacaggagaagagagaggaggaggagagctgtagctgctaagatttatactggtttaagagctcaaggaaactataatcttccgaagcactgtgataacaatgaagttcttaaagctctttgtactgaagctggttggatcgttgaacctgatggtaccacttatcgcaagggatgcaagccaaccccgatggagattggaggcacttcaacaaacatcacgccaagttcttcacggcatccaagtcccccatcatcatactttgctagcccaattccatcttatcagccaagtccaacttcctcttctttccccagtccatctcgtgctgatgccaacatgttatcacatccatattcttttctccaaaatgtcgttccttcatcccttcctccattacgaatatcaaacagtgcccctgtaactccacctctttcatcaccaactaggcatcctaagcaaactttcaatttagaaactttggccaaagaatcaatgtttgctttaaacatccctttctttgctgcttcagccccagcaagcccaactagggttcagcgttttactcctccaactatacccgagtgtgatgaatctgactcatctaccattgattcaggccagtggatcaactttcaaaagtatgcgtcaaatgttccaccttctccaacatttaatcttgtaaaacctgtgcctcagccgcttcgtcctaatgatatgatcacagacaagggtaagagcatagacttcgactttgaaaatgtatcagtcaaggcatgggaaggtgaaaggattcacgatgtaggattcgatgatctggaactcacacttggaagtggcaatgctcgcatatga

SEQ ID NO: 13 - protein, 322 aa, Petunia x hybrida
MTAAGGGGGSATGRLPTWKERENNKRRERRRRAIAAKIFTGLRAQGNFKLPKHCDNNEVLKALCIEAGWIVEDDGTTYRKGHRRPPIEYAVSSMDISACSSIQPSPMSSSFPSPVPSYHASPTSSSFPSPSRCDGNPSSYILPFLHNLASIPFSLPPLRISNSAPVTPPPSSPTRGSQPKPVWESFSRGPLHSFHHPIFAASAPSSPTRRQYSKPATIPECDESDAASVESARWVSFQTLAPSAAPTSPTFNLVKPVIPQQNILLDALSGRGAFGWGEAAQRGHASEFDLESCKVKAWEGERIHEVAVDDLELTLGAGKAHA

SEQ ID NO: 14 - DNA, 969 bp, Petunia x hybrida
atgacagctgccggcggtggaggtggatcagcaacggggagattaccaacatggaaagagagagaaaacaataagaggagagagagaagaagaagagctattgctgccaaaatatttacagggctaagagcacaaggtaacttcaaacttccaaaacactgtgataataatgaagtcttgaaagctctttgtattgaagctggttggattgttgaagatgatggcaccacttatcgcaagggacacaggcgtccaccaattgaatatgctgtttcctctatggatatcagtgcatgctcatcgattcagcctagcccaatgtcatcctctttccccagtcctgtaccttcttaccatgccagcccaacatcatcctcattccctagcccctcccgctgtgacggaaatccgtcgtcatacatcctcccctttctccataacttagcttccattccgttttctctgccacctcttcgtatatctaacagtgcccctgttacgccacccccttcttctcctacccgagggtcacagcccaaacctgtctgggaatccttctccaggggtccattacattctttccaccacccgattttcgctgcttctgcaccatcaagtcctacccgccgccaatactctaaacctgctacaattccagaatgcgatgagtctgatgctgcctcagttgagtctgcacgctgggtcagcttccagacgttggcaccttcagcagctccaacttcgcctacttttaaccttgtaaaacctgtcattcctcagcagaatattcttttagatgcattaagtggccgtggagcgtttggctggggtgaagcagctcaaaggggacatgcctctgaatttgaccttgaaagctgcaaagtgaaggcatgggaaggtgagagaatacatgaagttgctgtggatgatctagagctcactcttggggccggaaaggcacatgcttga

SEQ ID NO: 15 - protein, 311 aa, Cucumis sativus
MTSDGATSASNCRRKPSWRERENNRTRERRRRAIAAKIYSGLRAQGNFNLPKHCDNNEVLKALCAEAGWTVEDDGTTYRKGCKPPPIDIVGTSTKITPYSSQNPSPLSSSFPSPMGSYQVSPSSSSFPSPSRYDANNPSNLIPYLRQAIPISLPPLRISNSAPVTPPLSSPASRTPQPFPNWEVNAKESLSSLNYPFFAVSAPASPTRPQLHTPAPIHKCDESESSTNDSNQWALFRAYAPSASTMPTSPTFNLVKPADQHVLHGGFIQENGRRNEFEFLGYKVKPWEGEKIHDVGLEDLELTLGSSKARS

SEQ ID NO: 16 - DNA, 936 bp, Cucumis sativus
atgacctccgacggcgccacttctgcctccaattgtcgccgtaagccttcttggagggagagggagaataatcggaccagagagcgccggagaagagccatcgctgctaagatttactccggtcttagagctcagggtaacttcaatttgcctaaacactgtgacaataacgaggtccttaaagccctctgtgctgaagctggttggactgtcgaagatgacggaaccacttatcggaagggatgcaagccacctcccattgatattgttggcacttcaaccaaaatcaccccttattcttcccaaaatccaagcccattgtcttcttctttccccagtcctatgggatcctaccaagtaagtccctcctcctcctccttcccgagcccgtcccgttacgatgccaataatccgtccaatctcattccttatcttcgccaagcgattcccatttcgcttcctcctcttagaatctcaaacagtgctcctgtgacccctcccctttcatccccagcctcaaggactccgcagccattccccaattgggaagtcaatgctaaagaatccttgtcctctttgaattaccctttctttgcagtatctgctccagctagcccgacccgccctcagctccatactcccgccccgatccacaaatgtgatgaatctgaatcatctaccaatgattccaatcaatgggcacttttccgtgcctatgcgccttctgcatcgacaatgccaacatctcccactttcaaccttgtgaaacctgctgatcagcatgttctgcatggtggtttcatccaagaaaatggaagaagaaacgaattcgagttcttggggtacaaggttaagccttgggaaggggagaaaatccacgacgttggattggaggatttagagctcacacttggtagcagtaaggctcgaagctaa

SEQ ID NO: 17 - protein, 307 aa, Vitis vinifera
  misc_feature (260)..(260): Xaa is Glu or Gly
MTSERTPTRRKASWKERENNMRRERRRRAIAAKIYAGLRAQGNYRLPKHCDNNEVLKALCSEAGWTVEDDGTTYRKGCKPPPSTEIAGTSTNNTPCSSQKPSPPSSSFPSAFASYQPSPSSSNLSFMDANASLNLLPFLYKSIPSSLPPLRISNSAPVTPPISSPTSRVPMPKPNWESLAKESMASIHHHYPIFAASAPASPSRCQYIAPATIPEYEESDTSTVESGQWVSFQTFARHLAPLPPTFNLMKPVAQKISPDXATKEKGITPELEIGSAQVKPWEGERIHEIGLDDLELTLGSGKSRSKG

SEQ ID NO: 18 - DNA, 924 bp, Vitis vinifera
atgacgtcagagaggacgccgacgaggaggaaggcgtcgtggaaggagagggagaacaacatgaggagagagaggaggaggagagccatagcggcaaagatatatgcgggcctcagggctcagggcaactatcgtcttccaaaacactgcgataacaacgaggtcctcaaggctctctgctccgaagctggttggaccgttgaagatgacggcaccacctatcgcaagggatgcaagcctcccccctcaactgagattgcaggaacttccacaaacaacactccctgctcttcccagaaaccaagcccaccatcttcctcctttccaagcgcattcgcttcctaccaacccagtccctcatcctcaaacctgtctttcatggatgccaatgcctctctcaatctccttccatttctctacaagtctatcccttcatctctgcctcctctccgaatatcaaacagtgctcctgtaacaccacctatttcgtccccaacctccagagttcccatgcctaaacccaactgggagtcccttgccaaagaatccatggcctctatccatcaccattaccccatctttgctgcttctgccccagcaagcccttctcgctgtcagtatattgctcctgccactatacctgaatatgaggagtctgacacctcaactgttgagtcaggccagtgggtgagtttccagacgtttgcacgccatcttgctccattgcccccaaccttcaatcttatgaaacctgtggctcagaaaatttcaccagatgragcaaccaaagagaaggggataactcccgagttggaaattggaagtgcacaggtgaagccctgggaaggggagaggattcatgagataggtttggatgatctggagcttacactaggaagtggaaagagtaggagtaaaggctaa

SEQ ID NO: 19 - protein, 317 aa, Amygdalus persica
MTGGGSSGRLPTWKERENNKRRERRRRAIAAKIYSGLRAQGSYKLPKHCDNNEVLKALCAEAGWVVEEDGTTYRKGCKPSPMEIAGTPTNMSACSSIQQSPQSSAFPSPVPSYHASPSSSSFPSPTRFDGNPSSYLLPFLRNIASIPTNLPPLRISNSAPVTPPLSSPTSRGSKRKPDWDSLTNGCLNSLRHPLFAASAPSSPTRRHHLTPATIPECDESDASTVDSGRWVSFQTGAPSVAPPSPTFNLMKPVAEQSVLQNTINGHGGMTWGNTTERGRGSEFEFESGTLKAWEGERIHEVGVDDLELTLGNGKNHV

SEQ ID NO: 20 - DNA, 954 bp, Amygdalus persica
atgacagggggtgggtcgtcggggaggttaccaacatggaaagagagggagaacaacaagaggagggagaggaggagaagagccattgctgctaagatatactctggtctcagagctcagggcagctacaagcttcctaagcactgcgacaacaacgaggtcttgaaagctctctgtgctgaagctggctgggtcgttgaagaagatggcaccacataccgcaagggatgcaagccatctccaatggaaattgcaggcactccaacaaatatgagtgcatgttcctctattcaacaaagcccacaatcctcagctttcccaagtcctgtaccatcctaccatgccagtccatcctcctcttccttcccaagcccaactcgtttcgatggaaacccctcctcttaccttcttccgttcctgcgtaacatagcttccattcctacaaatcttcctcctcttagaatatccaatagtgcgcctgtaactccacctctttcttctccaacctctagaggttcaaagcgaaaacctgactgggactcccttactaatggctgtctaaactccttgcgccaccctcttttcgcagcttctgccccttcaagccctacgcgtcgccaccatcttacacctgccacaataccagaatgtgatgagtccgatgcttccactgtggactccggtcgttgggtcagtttccagacaggggcaccatcagtagctccaccttcacccacatttaatcttatgaaaccggtggctgagcagagtgttcttcagaacactattaatgggcatgggggaatgacctggggaaacactacagagaggggacgaggctcagaatttgagtttgagagcggcacactgaaagcttgggagggtgagagaatacatgaggtaggggtggatgatctggagcttacacttggaaatgggaagaaccatgtttaa

SEQ ID NO: 21 - protein, 317 aa, Populus trichocarpa
MTSDGATSTSAAMAAATRRKPSWRERENNRRRERRRRAIAAKIFTGLRAQGNYNLPKYCDNNEVLKALCAEAGWVVEEDGTTYRKGHRPPPIEIVGTSTRVTPYSSQNPSPLSSLFPSPIPSYQASPSSSSFPSPTRGDNNASSNLLPFLRSAIPLSLPPLRISNSAPVTPPLSSPTSRNPKPIPNWDFIAKQSMASFSYPFNAVSAPASPTHRQFHAPATIPECDESDTSTVESGQWISFQKFAPSVAAAMPTSPTYNLVIPVAQQISSSNLVKESAVPMDFEFGSEQVKPWEGERIHEVGLDDLELTLGSGKAQS

SEQ ID NO: 22 - DNA, 954 bp, Populus trichocarpa
atgacgtcagatggggcaacctcgacgtcagctgcaatggcggcagctacaaggaggaagccatcgtggagggagagggagaataataggaggagagagaggaggaggagagctatagctgcaaaaatatttactgggttaagggctcaagggaattataatttgcctaaatattgtgacaataatgaggtattgaaagctctctgtgctgaggctggttgggttgttgaagaagatggaactacttatcgcaagggacacaggccacctcctattgagatagtaggtacttcgacgagggtaaccccatactcatcccaaaatcctagtccactatcttcgttgtttcccagtccaattccttcctatcaagccagtccctcctcctcctcgtttcctagccctactcgtggcgataacaatgcctcttctaatctccttccattccttcgaagtgccattccattgtctcttcctcctcttcgaatctcaaacagtgcgcctgtaaccccacctctctcgtccccgacctcaagaaaccccaagccaattcccaactgggattttattgccaaacaatccatggcctcctttagttacccattcaatgcagtgtctgctccggctagcccaactcatcgtcaatttcatgctccagccactatacctgaatgtgatgagtctgatacatccactgtggagtctggtcagtggataagctttcaaaagtttgcgccttctgtggctgcagcaatgccaacctctcctacctataatcttgtgatacccgtggctcagcaaatttcgtctagcaatttggtcaaagagagtgcagtgcccatggattttgagtttggtagtgaacaggtgaaaccatgggaaggagagaggattcatgaagtaggattagatgatctagagctcacacttggaagtggcaaggctcagagttag

SEQ ID NO: 23 - protein, 304 aa, Oryza sativa
GTELVGMTSGAAAAGRTPTWKERENNKRRERRRRAIAAKIFTGLRALGNYNLPKHCDNNEVLKALCREAGWVVEDDGTTYRKGCKPPPSSAGGASVGMSPCSSTQLLSAPSSSFPSPVPSYHASPASSSFPSPSRIDNPSASCLLPFLRGLPNLPPLRVSSSAPVTPPLSSPTASRPPKIRKPDWDVDPFRHPFFAVSAPASPTRGRRLEHPDTIPECDESDVSTVDSGRWISFQMATTAPTSPTYNLVNPGASTSNSMEIEGTAGRGGAEFEFDKGRVTPWEGERIHEVAAEELELTLGVGAK

SEQ ID NO: 24 - DNA, 915 bp, Oryza sativa
gggacggagctggtgggtatgacgtccggggcggcggcggcggggaggacgccgacgtggaaggagagggagaacaacaagaggcgggagcggcggcggcgtgccatcgccgccaagatcttcacggggctccgggcgctcgggaactacaacctccccaagcactgcgacaacaacgaggtgctcaaggcgctctgccgcgaggccggctgggttgtcgaggacgacggcaccacctaccgcaagggatgtaagccgccgccatcgtcggctgggggagcgtcggtggggatgagcccctgctcgtcaacgcagctgctgagcgcgccgtcgtcgtcgttcccgagcccggtgccgtcgtaccacgcgagcccggcgtcgtcgagcttcccgagccccagccggatcgacaacccgagcgcctcctgcctcctcccgttcctccgggggctccccaacctcccgccgctccgcgtctccagcagcgcgcccgtcacgccgccgctctcgtcgccgacggcgtcgcggccgcccaagatcaggaagccggactgggacgtcgacccgttccggcaccccttcttcgcggtctccgcgccggcgagccccacccgcggccgccgcctcgagcacccggacacgataccggagtgcgacgagtccgacgtctccacggtggactccggccggtggatcagcttccagatggccacgacggcgccgacgtcgcccacctacaacctcgtcaacccgggcgcctccacctccaactccatggagatagaagggacggccggccgaggcggcgcggagttcgagttcgacaaggggagggtgacgccatgggagggcgagaggatccacgaggtcgccgccgaggagctcgagctcacgctcggcgtcggcgcgaaatga

SEQ ID NO: 25 - protein, 312 aa, Triticum aestivum
MTSGAARAAAAAEAEAGLGRTPTWKERENNKRRERRRRAIAAKIFTGLRALGNYKLPKHCDNNEVLKELCREAGWVVEDDGTTYRKGYKPPSSGPFGGVSSAGMSPCSSSQLLSAPSSSFPSPVPSYHASPASSSFPSPTRLDNPSPACLLPFLRGLPLPPLRVSNSAPVTPPLSSPTASRPPKILKPDWEVDPFRHPFFALSAPASPTRGRRHEHPDTIPECDESDVSTVDSGRWISFQMATTAPTSPAYNLVNLGASSSNSMEMEGMAGERGRSGPEFEFDKGRVTPWEGERIHEVAAEELELTLGVGSK

SEQ ID NO: 26 - DNA, 939 bp, Triticum aestivum
atgacgtcgggggcggcccgggcggcggcggcggcggaggcggaggccgggctggggcggacgcccacgtggaaggagcgggagaacaacaagcgccgcgagcgccggcgccgggccatcgccgccaagatcttcaccggcctccgcgcgctcggcaactacaagctgcccaagcactgcgacaacaacgaggtgctcaaggagctctgccgcgaggccggctgggtcgtggaggacgacggcaccacctaccgcaagggatacaagccgccgtcgtccgggccgttcggtggggtctcgtcggcgggcatgagcccctgctcgtcctcgcagctgctcagcgcgccctcgtcgtcgttcccgagcccggtgccttcctaccacgccagcccggcgtcgtcgagcttcccgagccccacgcgcctcgacaacccgagccccgcctgcctcctcccgttcctccgtggcctccccctgcccccgctccgggtctccaacagcgcgccggtgacgccgccgctctcttcgccgacggcgtcgcggccgcccaagatcctgaagccggactgggaggtggacccgttccggcacccgttcttcgccctctccgcgccggctagccccacccgtggccgccggcacgagcacccggacacgataccggagtgcgacgagtcagacgtctccacggtggactctggccggtggatcagcttccagatggccaccacggcgccgacctcgcccgcgtacaacctcgtcaacctaggcgcctccagctccaactccatggagatggagggaatggcgggggagcggggccgaagcggtccggagttcgagttcgacaaggggagggtgacgccgtgggaaggggagaggatccacgaggtcgccgccgaggagcttgagctcacgctcggcgtcggctcgaaatga

SEQ ID NO: 27 - protein, 313 aa, Aegilops tauschii
MTSGAARAAAAAEADAGLGRTPTWKERENNKRRERRRRAIAAKIFTGLRALGNYKLPKHCDNNEVLKELCREAGWVVEDDGTTYRKGYKPPSSGPFGGVSSAGMSPCSSSQLLSAPSSSFPSPVPSYHASPASSSFPSPSRLDNSSPACLLPFLRGLPNLPPLRVSNSAPVTPPLSSPTASRPPKILKPDWEVDPFRHPFFALSAPASPPRGRRHEHPDTIPECDESDVSTVDSGRWISFQMATTAPTSPAYNLVNLGASSSNSMEMDGMAGERGRSGPEFEFDKGGGTPWEGERIHEVAAEELELTLGVGSK

SEQ ID NO: 28 - DNA, 939 bp, Aegilops tauschii; identical to SEQ ID NO: 26

SEQ ID NO: 29 - protein, 317 aa, Hordeum vulgare
MTSGAARAAAAAAAAAEADAGLGRTPTWKERENNKRRERRRRAIAAKIFTGLRALGNYKLPKHCDNNEVLKELCREAGWVVEDDGTTYRKGYKPPSSGPFGGVSSAGMSPCSSSQLLSAPSSSFPSPVPSYHASPASSSFPSPTRLDNPSPACLLPFLRGLPNLPPLRVSNSAPVTPPLSSPTASRPPKILKPDWEVDPFRHPFFALSAPASPTRGRRHEHPDTIPECDESDVSTVDSGRWISFQMATTAPTSPAYNLVNLGASSSNSMEMEGMAGERGRGGPEFEFDKGRVTPWEGERIHEVAAEELELTLGVGSK

SEQ ID NO: 30 - DNA, 954 bp, Hordeum vulgare
atgacgtcgggggctgcccgggcggcggcggcggcggcggcggctgcggaggcggacgccgggctggggcggacgccgacgtggaaggagcgggagaacaacaagcggcgcgagcggcggcgccgggccatcgccgccaagatcttcaccggcctccgcgcgctcggcaactacaagctccccaagcactgcgacaacaacgaggtgctcaaggagctctgccgcgaggccggatgggtagtggaggacgacggcaccacctaccgcaagggatacaagccgccgtcgtccgggccgttcggtggggtctcgtcggcgggcatgagcccctgctcgtcctcgcagctgctcagcgcgccgtcgtcgtcgttcccgagcccggtgccttcctaccacgccagcccggcctcgtcgagcttcccgagccccacgcgcctcgacaacccgagccccgcctgcctcctcccgttcctccgtggcctccccaacctgcccccgctccgggtctccaacagcgcgccagtgacgccgccgctctcgtcgccgacggcgtcgcgcccgcccaagatcctgaagcccgactgggaggtcgacccgttccggcacccgttcttcgccctctccgcgccggcgagccccacccgtggccgccggcacgagcatccggacacgataccggagtgcgacgagtcggacgtctccacggtggactctggccggtggatcagcttccagatggccacgacggcgccgacgtcccccgcgtacaacctcgtcaacctaggcgcctccagctcaaactccatggagatggagggaatggcgggggagaggggccgaggcggcccggaattcgagttcgacaaggggagggtgacgccgtgggaaggggaaaggatccatgaggtcgccgccgaggagcttgagctcacgctcggcgtcggctcaaaatga

SEQ ID NO: 31 - protein, 317 aa, Zea mays
MTSGAAAAGGLGRTPTWKERENNKRRERRRRAIAAKIFTGLRALGNYKLPKHCDNNEVLKALCREAGWVVEDDGTTYRKGCRPPPGMLSPCSSSQLLSAPSSSFPSPVPSYHASPASSSFPSPTRLDHSSGGSSTHNPAAAAAAAAASLLPFLRGLPNLPPLRVSSSAPVTPPLSSPTAAAASRPPTKVRRPNWDAAAAVVAADPFRHPLFAVSAPASPTRARRREHPDTIPECDESDVCSAVDSARWISFQATTAPASPTYNLVHPASDSMELDGTTAAVEEFEFDKGRVVTPWEGERIHEVAAEELELTLGVGAK

SEQ ID NO: 32 - DNA, 954 bp, Zea mays
atgacgtccggggcggcggcggcgggaggtctggggcggacgccgacgtggaaggagcgggagaacaacaagcgccgggagcgccggcggagggccatcgccgccaagatcttcacgggcctccgcgccctcggcaactacaagctgcccaagcactgcgacaacaacgaggtgctcaaggcgctctgccgcgaggcggggtgggtcgtcgaggacgacggcaccacctaccggaagggttgcaggccgccgccggggatgctgagcccgtgctcgtcgtcgcagctgctgagcgcgccgtcctcgagcttcccgagcccggtgccgtcctaccacgccagcccggcgtcgtcgagcttcccgagcccgacgcgcctcgaccacagcagcggcggcagcagcacccacaaccccgccgcggcggccgcggcggccgccgcctccctgctcccgttcctccggggcctgccgaacctgccgccgctccgcgtgtccagcagcgcgcccgtcacgccgccgctctcctcgcccacggcggcggcggcgtcgcggccgcccaccaaggtccgcaggcccaactgggacgccgccgccgccgtcgtcgccgctgaccccttccggcaccccttgttcgcggtctccgcccccgccagccccacccgcgcgcgccggcgcgagcacccggacaccatcccggagtgcgacgagtccgacgtctgctccgcggtcgactccgcccggtggatcagcttccaggccaccacggcgcccgcgtcgcccacgtacaacctcgtccacccggcctccgactccatggagctggacgggacgacggcagccgtcgaggagttcgagttcgacaagggccgcgtcgtcacgccgtgggaaggcgagcggatccacgaggtcgccgccgaggagctcgagctcacgctcggcgtcggcgccaagtga

SEQ ID NO: 33 - protein, 336 aa, Sorghum bicolor
MTSGAAAAAAAAGALGRTPTWKERENNKRRERRRRAIAAKIFTGLRALGNYKLPKHCDNNEVLKALCREAGWVVEDDGTTYRKGCKPPPGMMSPCSSSQLLSAPSSSFPSPVPSYHASPASSSFPSPTRLDHSSGSNHHHHHNPGPTAAAAAAAASSLLPFLRSLPNLPPLRVSSSAPVTPPLSSPTAASRPPTKVRKPDWDAAVADPFRHPFFAVSAPASPTRARRREHPDTIPECDESDVCSTVDSGRWISFQVGAATTAPASPTYNLVHPAGGGASASNSMELDGMAAADIGGRGGGPAEFEFDKGRVTPWEGERIHEVAAEELELTLGVGAK

SEQ ID NO: 34 - DNA, 1011 bp, Sorghum bicolor
atgacgtcgggggcggccgcagcggcggcggcggcgggagcgctgggtcggacgccgacgtggaaggagcgggagaacaacaagcgccgggagcgccggcggagggccatcgccgccaagatcttcacgggcctccgcgcgctcggcaactacaagctgcccaagcactgcgacaacaacgaggtgctcaaggcgctctgccgcgaggcgggctgggtcgtcgaggacgacggcaccacctaccgaaagggatgcaagccgccgccggggatgatgagcccgtgctcgtcctcgcagctgctgagcgcgccgtcgtcgagcttcccgagcccggtgccgtcctaccacgccagcccggcgtcatccagcttcccgagcccgacgcgcctcgaccacagcagcggcagcaaccaccaccaccaccacaacccgggccccactgctgccgctgccgcggcggcggcctcctccctgctcccgttcctccggagcctgccgaacctcccgccgctccgcgtctccagcagcgcgcccgtcacgccgccgctctcctcgcccacggcggcgtcgcggccgcccaccaaggtccgcaagcccgactgggacgccgccgtcgccgacccgttccggcaccccttcttcgcggtctccgcccccgccagccccacccgcgcacgccggcgcgagcacccggacaccatccccgagtgcgacgagtccgacgtctgctccaccgtcgactccggccgctggatcagcttccaggtgggcgcggcgaccacggcgcccgcgtcgcccacctacaacctcgtccacccggccggcggcggcgcgtccgcctccaactccatggagctggacgggatggcggccgcggacatcggcggcaggggcggcggtcccgcggagttcgagttcgacaagggccgtgtcacgccgtgggaaggcgagcgcatccacgaggtcgccgccgaggagctcgagctcacgcttggcgtcggcgccaagtga

SEQ ID NO: 35 - protein, 346 aa, Brachypodium distachyon
MTSGAARAAGEAAAGDGGLGRTPTWKERENNKRRERRRRAIAAKIFTGLRALGNYKLPKHCDNNEVLKELCREAGWVVEDDGTTYRKGYKPPPSSGPFGGSGVSSAGMSPCSSSQLLSAPSSSFPSPVPSYHASPASSSFPSPNRLDTTNNLNHHPASAAACSLLPFLRGLPNLPPLRVSNSAPVTPPLSSPTSSSSRPPKIQKQGGWGGDAEYPFRHPFFALSAPASPTRGRRHEHPDTIPECDESDVSSTVDSGRWISCQMAATTAPTSPAYNLVNNNNNSNRGGGASGSSSMEIEGMMAAAGERGRGGAEFEFDKGRVTPWEGERIHEVAAEELELTLGVGAK

SEQ ID NO: 36 - DNA, 1041 bp, Brachypodium distachyon
atgacgtcgggggcggcgcgggcagcgggggaagcggcggcgggggacggcgggctggggcggacgccgacgtggaaggagcgggagaacaacaagcggcgggagcggcggcgccgggccatcgccgccaagatcttcacgggcctccgcgcgctcggcaactacaagctccccaagcactgcgacaacaacgaggtgctcaaggagctctgccgcgaggccggctgggtcgtcgaggacgacggcaccacctaccgcaagggatacaagccgccgccgtcgtcggggccgttcggtggcagcggggtgtcgtccgcggggatgagcccctgctcgtcctcgcagctcctgagcgcgccgtcctcgtcgttcccgagcccggtgccgtcctaccacgcgagcccggcttcctccagcttcccgagccccaatcgcctcgacaccaccaacaacctcaaccaccatcccgcgagcgccgccgcctgcagcctcctcccgttcctccggggcctcccgaacctgcccccgctgcgcgtctccaacagcgcccccgtcacgccgccgctctcctcgcccacgtcctcctcctcccggccccccaagatccagaagcagggcggctggggcggcgacgccgagtacccgttccggcaccccttcttcgcgctctccgcgcccgccagccccacccgcggccgccgccacgagcacccggacaccatccccgagtgcgacgagtcggacgtctcctccaccgtcgactccggccgctggatcagctgccagatggcggccaccacggcgcccacgtccccggcatacaacctcgtcaacaacaacaacaacagcaaccgaggaggcggcgcgtctgggtccagctccatggagatcgaggggatgatggcggcggcgggggagaggggccgtggaggagcggagttcgagttcgacaaggggagggtcacgccgtgggaaggggagaggatccatgaggtcgcggccgaggagctcgagctcacgctcggcgtcggcgcgaaatga

SEQ ID NO: 37 - protein, 367 aa, Picea sitchensis
MTSGSRLPTWKERENNKKRERRRRAIAAKIYAGLRMYGNYKLPKHCDNNEVLKALCAEAGWMVEEDGTTYRKGCKPTERIEVAGSSSVSPASSYHPSPAPSYQPSPASSSFASPASSSFEPAGTGAANSLIPWLKNLSSSSSASSSGRLIHGGGSISAPVTPPLSSPTGRGARAKLDWDAMVKAVANESNDCPNSGFSTPVSPWSNYPFVASSTPASPGRHAEMATQLSNAVVDKGRWMGGIRMMAFPSAGPSSPTFNLLTPAAQLQHGLATEGGRLWTPGQSGVSSPCNNRAGEEERLLPPFQEGMDASDEFAFGSVAVKPWQGERIHEECGGEIGSDDLELTLGSFSSSSSKLRSDREPLFSVKE

SEQ ID NO: 38 - DNA, 1104 bp, Picea sitchensis
atgacgtctggttcgaggctgccgacctggaaagaacgggagaataacaagaagagagagcggcgaaggcgagcgatcgcggctaagatctatgccgggcttcggatgtatggaaactacaagctgccgaagcattgcgataacaacgaggttctcaaggccctgtgtgcagaagccggctggatggtcgaggaagatggaacaacttaccgcaagggatgcaagccgacggagcgcatagaggtcgcgggatcgagttccgttagcccggcgtcttcttatcatccgagcccggcgccttcctaccagcccagccctgcgtcgtcgtcgttcgcgagcccggcttcgtcatcgttcgagcccgcgggaaccggggcggcgaattctttgatcccgtggctcaagaacctctcgtcgtcgtcgtcggcgtcttcttcgggccggctgattcacggcgggggctcgatcagcgcgccggtgactccgcctctgagctccccaacaggccgcggcgcgcgggcgaagctggactgggacgccatggttaaagccgttgccaatgagagcaatgattgtcctaattcggggttctctacccccgtgagcccctggtcgaattaccccttcgtggcgtcctccaccccggcgagcccggggcgtcacgccgagatggccacgcagttgagcaacgccgtggtggacaaggggcgctggatgggcgggatccggatgatggcgttcccctcggcggggccttcgtcgcccacgttcaatctgctgaccccggccgcgcagcttcagcacggccttgcaacggagggcggcaggctgtggacgccggggcagagtggcgtctcgtctccttgtaacaaccgggcaggtgaggaggagaggttattgccgccgtttcaagaaggtatggatgcttcagacgagtttgctttcggcagcgttgcggtcaagccgtggcaaggcgagaggattcatgaggagtgcgggggagagataggatccgacgacctagagcttacgctaggatctttctcgtcatcttcttcaaagttaagatctgaccgagaacccttgttttctgttaaagagtag

SEQ ID NO: 39 - DNA, 24 bp, Artificial
  primer
caccatgacttcggatggagctac

SEQ ID NO: 40 - DNA, 20 bp, Artificial
  primer
tcaaccacgagccttcccat

SEQ ID NO: 41 - DNA, 20 bp, Artificial
  primer
cgacacacttgtctactcca

SEQ ID NO: 42 - DNA, 20 bp, Artificial
  primer
cccaaccagcttcaacacaa